(19) United States
(12) Patent Application Publication
(10) Pub. No.: US 2009/0252842 A1
(43) Pub. Date: Oct. 8, 2009

(54) APPARATUS, SYSTEMS, AND METHODS OF EXTENDING USEFUL LIFE OF FOOD TREATING MEDIA BY INHIBITING DEGRADATION THEREOF

(75) Inventors: DanLi . Wang, Shoreview, MN (US); Travis B. Hoium, Minneapolis, MN (US); John J. Dyer, Shoreview, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 12/412,841

(22) Filed: Mar. 27, 2009

Related U.S. Application Data
(60) Provisional application No. 61/042,477, filed on Apr. 4, 2008.
(60) Provisional application No. 61/106,313, filed on Oct. 17, 2008.
(60) Provisional application No. 61/147,266, filed on Jan. 26, 2009.
(60) Provisional application No. 61/158,102, filed on Mar. 6, 2009.

(51) Int. Cl. G01N 33/03; A23L 3/32; A47J 37/12

(52) U.S. Cl. 426/231; 426/244; 99/330; 99/358

(57) ABSTRACT

Apparatus, systems, and methods of extending useful life of food treating media by inhibiting its degradation through application of electrons are disclosed. Application may be automatic and responsive to monitoring quality parameters of a medium. The apparatus, systems, and methods are retrofittable to existing food treating apparatus.

### CROSS REFERENCE TO RELATED APPLICATIONS

This application claims the benefit of U.S. Provisional Patent Application No. 61/042,477, filed Apr. 4, 2008; U.S. Provisional Patent Application No. 61/106,313, filed Oct. 17, 2008; U.S. Provisional Patent Application No. 61/147,266, filed Jan. 26, 2009; and U.S. Provisional Patent Application No. 61/158,102, filed Mar. 6, 2009, the disclosures of which are incorporated by reference herein in their entirety.

### BACKGROUND

The present description is directed to apparatus, systems, and methods regarding the use of food treating media and, more particularly, to apparatus, systems, and methods of efficiently and economically extending the useful life of food treating media, such as cooking oils and fats, in different cooking environments.

Degradation of food treating media during food treating is widely recognized. Deep frying is one example of an extremely popular way of treating or preparing foods, and is typically a source of excessive levels of volatile and nonvolatile decomposition products, such as free fatty acids, total polar components (TPC), and acrylamides due primarily to overuse and/or overheating of the cooking oils, fats and carbohydrates. Excessive levels of these volatile and nonvolatile decomposition products have been associated with several kinds of diseases, such as hypertension, heart attacks, and diabetes. Free fatty acids, total polar components (TPC), and acrylamides tend to build-up in, for example, cooking oils and fats when subjected to, for example, oxidation and hydrolysis. Oxidation and/or hydrolysis tend to increase over prolonged periods of cooking oil use, especially when overheated.

Typically, overuse and overheating of the same oil batch in a fryer vat tend to lessen oil stability and thus, its useful life. Presently, there are trends for replacing partially hydrogenated vegetable oil with trans-fat free vegetable oil due to health reasons. However, the latter type of oil is less stable during usage and more costly. Presently, millions of tons of oil and fats are used worldwide for deep frying. Moreover, there is a significant amount of presently existing hardware, such as fryers, in use that handle such oil and fats.

Efforts have been directed to extending the useful life of cooking fats and oils while maintaining the good oil quality by reducing the amount of decomposition components. For example, private and governmental efforts have sought to reduce excessive build-up of decomposition products, such as free fatty acids and total polar components (TPC). Some efforts have led governments to impose restrictions on the amounts of decomposition products in cooking oils and foods. Higher standards may present economic issues for establishments with existing equipment, such as deep frying equipment, trying to rapidly comply with newer regulations, standards, etc. because of the potential significant investments of capital for newer equipment and/or more expensive oils that would otherwise be required in order to comply. Moreover, by extending the useful life of the cooking oils considerable savings are also realized insofar as replacement volumes of unhealthy cooking oils are diminished.

Accordingly, there is a desire to easily and economically retrofit equipment with inexpensive approaches that may extend the useful life of cooking oils. In addition, Moreover, there is desire to provide for highly reliable and economical approaches for enhancing stabilization of cooking media quality, particularly ones which can be used on-line and on a real-time basis without protracted delays and unnecessary costs.

A common approach for preventing use of degraded cooking oil is to monitor, filter and replace it. Monitoring the quality of cooking oils typically relies upon workers replacing the oil based on their subjective judgments with respect to when the oil is degraded. Considering the impetus of the noted private and governmental efforts, there is a desire to minimize or remove subjective judgments of workers opining about the quality of the cooking oils. For example, cooking oils may be replaced if their color changes. However, for a worker determining at what point a change in color triggers replacement is problematic given the highly subjective nature of determining the adequacy of color changes. This issue is compounded given that there are various kinds of color changes that may arise from different kinds of cooking oils and foods prepared. Clearly, replacing cooking oil prematurely may result in wastage of otherwise costly and usable oil. On the other hand, using degraded oils containing excessive free fatty acids and total polar components (TPC) is unhealthy and may be in violation of applicable standards, rules, regulations, and laws.

Several other known methods for evaluating oil quality include monitoring chemical and physical parameters of the cooking oil. For example, some approaches use dielectric constant measurements, visible and infrared spectroscopes, Fourier transform infrared (FTIR), column chromatography, and ultrasonic techniques. Absorptive membranes and surface acoustic waves (SAW) have also been used to measure oil quality. Many of the foregoing methods, while minimizing or reducing subjective judgments about oil quality, are, however, tedious and time consuming. Some send oil samples to remote labs for testing. To minimize drawbacks and delays some efforts have proposed real-time monitoring of cooking oils with optical probes or with measurements obtained by measuring dielectric responses of the cooking media.

Besides monitoring oils and fats in order to determine when they should be replaced, other approaches have extended the useful life of the cooking oils and fats before replacement. Extending useful life has included using costlier higher quality fats and oils that operate to slow degradation caused by oxidation. While this latter approach is sound, it nevertheless requires relatively costlier oils.

Other efforts to extend useful life include minimizing thermal degradation by use of thermal controls for preventing overheating of cooking oils and fats. While such approaches are satisfactory, they, nevertheless, may make retrofitting existing fryers costly, thereby forcing food establishments to consider buying newer and more expensive equipment.

Still other approaches to extend the useful life of cooking oils and fats include the common practice to filter the particulate food matter from the cooking oil to minimize the carmelization of such food matter within the cooking oil. Some conventional fryers utilize a batch filtration system in which the cooking oil is drained from the fryer vat and then manually or mechanically filtered before returning the cleansed cooking oil back to the fryer vat.

Other conventional fryers utilize a continuous filtration system, in which a filter is placed within a fluid path of the cooking oil, so as to continuously filter the cooking oil as it is being re-circulated between the fryer vat and the pump/heat exchanger. Clearly, adding such filtration systems to fryers may present significant retrofitting issues.

Still other approaches for extending the useful life of cooking oils include electrical systems for supplying electrons to the cooking medium. For example, one approach supplies electrons at a fixed rate directly to a cooking vessel. The cooking vessel itself acts as a cathode to a circuit that is integral with the vessel and includes an electrochemical battery. Not only is the rate fixed but also the amount of electron flow is limited because the electrochemical battery is relatively small and self-contained. Such an approach may not be suitable for robust commercial applications. Further, such approach requires food to be present during application of electrons during frying. Also, the battery and circuit must be a part of the cooking vessel. Furthermore, with such an approach there is no monitoring of the cooking oil in order to determine when to initiate operation of the battery.

Another approach uses an electronic probe that is suspended in the cooking oil and emits electrons at a fixed rate to the cooking medium. The probe may be entirely suspended in the fluid to inhibit oxidation of the cooking medium. This approach uses an electrode surrounded by an insulator and emits the electrons from a metal casing surrounding the insulator. However, the amount of oxidation may be limited by a fixed flow of electrons and the fact that it may be entirely suspended in the cooking oil. Also, with such an approach there is no monitoring of the cooking oil in order to determine when to initiate operation of the probe.

While there are successes using the former approaches for monitoring cooking oils or extending their useful life, none of these approaches have done so in a manner that effectively, economically, extends quality based on real-time assessments of oil quality during food treating, and in a manner that can be effectively and economically retrofitted to existing food treating systems, such as fryers or the like.

### SUMMARY

The present description includes a method of inhibiting degradation of a food treating medium. The method comprises: providing a container for containing a food treating medium and having at least a conductive portion; and, providing a low voltage source including a semiconductor material associated with the conductive portion having a food treating medium in contact with the conductive portion.

The present description includes having the above method provided low voltage source in contact with the conductive portion and at least partially immersed in a food treating medium contained in the container. The present description includes the above method wherein the applying being performed by the low voltage source is independent of the container. The present description includes the above method wherein the low voltage source is electrically connected to an exterior surface of the container. The present description includes the above method wherein a predetermined monitored value is obtained from monitoring a quality parameter of the food treating medium. The present description includes the above method wherein the providing the low voltage source includes applying electrons automatically responsive to the predetermined monitored value.

The present description includes a system of inhibiting degradation of a food treating medium. The system comprises a container for containing a food treating medium and having at least a conductive portion; and, one or more sources of electrons associated with the conductive portion for applying electrons from a surface of the conductive portion to a food treating medium in contact with the conductive portion.

The present description includes the above system wherein the one or more sources are engaged with an exterior surface of the container. The present description includes the above system wherein the one or more sources are in contact with the conductive portion and are also immersed in a food treating medium contained in the container. The present description includes the above system wherein the one or more sources are operable to apply electrons at a fixed rate and are operable of applying a variable rate. The present description includes the above system further comprising a monitoring assembly that monitors quality of the food treating medium and generates at least a signal related to food medium treating quality. The present description includes the above system wherein the one or more sources are automatically responsive to the predetermined value. The present description includes the above system wherein the one or more sources include using a semiconductor material.

The present description includes a method of inhibiting degradation of a food treating medium. The method comprises: providing a container having one or more vats for containing a food treating medium and having at least a conductive portion to each of the one or more vats; and, applying electrons by electrically coupling the conductive portion with one or more sources of electrons, independent of the container, for applying electrons from a surface of the conductive portion to a food treating medium in contact with the conductive portion of the one or more vats for inhibiting degradation of a food treating medium.

The present description includes a method of inhibiting degradation of a food treating medium. The method comprises: providing a container for containing a food treating medium and having at least a conductive portion; and, applying electrons by electrically coupling the conductive portion with one or more sources of electrons for applying electrons from a surface of the conductive portion to a food treating medium in contact with the conductive portion, wherein the one or more sources are separate and apart from a food treating medium contained in the container.

The present description includes a food treating method. The method comprises monitoring at least a quality parameter of a food treating medium to obtain a predetermined value correlated to quality; and applying electrons to the food treating medium in response to the predetermined value so as to inhibit degradation of the food treating medium.

The present description includes having that last noted method do the applying automatically responsive to the predetermined value. The present description includes having that last noted method of applying use one or more sources in conductive relationship with a vessel, further wherein the applying immerses the one or more source in the food treating medium. The present description includes having that last noted method wherein the predetermined value includes at one of the following group of parameter monitoring assemblies consisting of luminescent measurements, dielectric constant measurements, visible and infrared spectroscopy measurements, Fourier transform infrared (FTIR) measurements, column chromatography measurements, temperature measurements, density measurements, viscosity measurements, smoke measurements, e-nose measurements, and ultrasonic measurements. The present description includes having that last noted method wherein the monitoring includes monitoring the food treating medium at a temperature at which food is being treated and subjected to oxidation and hydrolysis. The present description includes having that last noted method wherein oxidation and hydrolysis of the food treating medium are altered by applying electrons at a different rate. The present description includes having that last noted method wherein the applying is by using one or more semiconductor materials.

The present description includes a food treating system. The food treating system comprises: a food treating apparatus configured for holding a food treating medium; a monitoring assembly for monitoring at least a quality parameter of a food treating medium to obtain a predetermined value correlated to quality; and a stabilizing assembly operable for applying electrons to a food treating medium in response to the predetermined value so as to inhibit degradation of the food treating medium.

The present description includes having that last noted system wherein the stabilizing assembly is automatically operative in response to the predetermined value.

The present description includes having that last noted system include one of the following group of parameter monitoring assemblies consisting of luminescent measurements, dielectric constant measurements, visible and infrared spectroscopy measurements, Fourier transform infrared (FTIR) measurements, column chromatography measurements, temperature measurements, density measurements, viscosity measurements, smoke measurements, e-nose measurements, and ultrasonic measurements. The present description includes having that last noted system wherein the predetermined value is storable in memory. The present description includes having the last noted above system include using a semiconductor material.

The present description includes a system that comprises: a vessel that includes at least a wall having a portion for defining at least a first chamber and a second chamber divided by a common wall for holding cooking oil; a probe supporting assembly mountable on the common wall for supporting a low voltage probe in each of the first and second chambers so that each of the probes are within the cooking oil, wherein the probe supporting assembly supports the probes at a position below a container that is insertable into at least of one the first and second chambers.

The present description includes a probe assembly for applying electrons to a cooking oil medium in a vat defined, in part, by a supporting wall including an electrically conductive portion. The probe assembly comprises: a housing assembly; a semiconductor material within the housing assembly for supplying electrons in response to energization thereof, wherein the semiconductor material is in electrically conductive relationship with at least a portion of the housing assembly for transferring electrons thereto; and, a coupling assembly on the housing assembly for securing and supporting the housing assembly on a supporting wall, whereby the electron transferring portion is in intimate engagement with the conductive portion of the supporting wall.

The present description includes a system adapted for mounting a plurality of probe assemblies relative to a fryer having a plurality of vats, the system comprises: a plurality of probe assemblies, a supporting assembly for supporting each of the probe assemblies such that one or more the probe assemblies cooperates with at least a respective one of the vats, the supporting assembly is attachable to the fryer; and, the probe assemblies are coupled to the supporting assembly.

The present description includes the last noted above system wherein a controller is operatively associated with each of the one or more probe assemblies and coupled to the probe assemblies through the supporting assembly for controlling each one of the probe assemblies. The present description includes the above system wherein the supporting assembly is a conduit to which each of the probe assemblies is coupled and through which each of the one or more probe assemblies is in electrical communication with the controller.

The present description includes a method of controlling a plurality of probe assemblies that are adapted to be associated with a fryer system containing a plurality of vats and into which different cooking oil media may be added, the method comprising: providing a plurality of probe assemblies each of which is independently operated; supporting each of the probe assemblies on the fryer so that one or more of the probe assemblies is operatively associated with at least one of the vats; and controlling operation of the probe assemblies.

The present description includes a method of inhibiting degradation of a food treating medium. The method comprises: providing a container for containing a food treating medium and having at least a conductive portion; and, providing a low voltage source of electrons integrated in the container and associated with the conductive portion and having a food treating medium in contact with the conductive portion.

The present description includes a system of inhibiting degradation of a food treating medium. The system comprises: a container for containing a food treating medium and having at least a conductive portion; and, one or more sources of electrons integrated in the container and associated with the conductive portion for applying electrons from a surface of the conductive portion to a food treating medium in contact with the conductive portion.

The present description includes an installation system for retrofitting a food treating system having one or more vats having sidewalls. The installation system comprises: a supporting assembly including a generally horizontally disposable supporting member that is adapted to be supported by a vat, and at least one or more generally extending supporting members arranged for depending in a generally upstanding relationship to and along the length of the horizontally disposable supporting member in response to the horizontally disposable supporting member being supported on a vat, wherein each of the one or more extending supporting members is coupled to the horizontally disposable supporting member at one end thereof, and at least one probe assembly coupled to another end of the one or more extending supporting members and oriented to be engageable with a vat sidewall.

The present description includes the above installation system wherein the at least one probe assembly is coupled to the extending supporting member that is rotatable about a generally elongated axis thereof, such that rotation of the extending supporting member allows the at least one probe assembly to be rotated for engaging different sidewalls of a vat. The present description includes the above installation system wherein the rotatable supporting member is coupled to the extending supporting assembly and able to be held stationary in at least one rotational position relative thereto so as to retain the probe assembly in the at least one rotational position in engagement with a vat sidewall. The present description includes the above installation system wherein the rotatable extending supporting member is coupled to the supporting assembly to seal against vapor and fluid.

The present description includes the above installation system further including a retaining assembly or retaining the at least one probe assembly in engagement with a vat sidewall. The present description includes the above installation system wherein the rotatable supporting member is coupled to the extending supporting assembly and able to be held stationary in at least one rotational position relative thereto so as to retain the probe assembly in the at least one rotational position in engagement with a vat sidewall. The present description includes the above installation system further including a retaining assembly for retaining the at least one probe assembly in engagement with a vat sidewall. The present description includes the above installation system wherein the supporting assembly is supported to be rotatable about its axis such that in response to it being rotated the at least one probe assembly is correspondingly rotatable away from engagement with a vat sidewall. The present description includes the above installation system wherein the supporting assembly includes a mounting bracket mountable on a wall of the food treating system so that the one probe assembly is in intimate engagement with at least one of the vat internal walls.

The present description includes the above installation system wherein the mounting bracket has a clamp mountable on a partition wall of a vat defining the one vat internal wall, and wherein the mounting bracket includes a pair of spaced apart projecting elements that receivably hold therebetween the horizontally disposable supporting member in a manner such that the one probe assembly is engageable with the one vat internal wall.

The present description includes the above installation system wherein the projecting elements enable the horizontally disposable supporting member to be rotatable while mounted thereon, whereby the probe assembly is rotatable away from engagement with the one vat internal wall. The present description includes the above installation system wherein at least one of projecting elements cooperates with the horizontally disposable supporting member to limit displacement of the horizontally disposable supporting member along its longitudinal axis while mounted. The present description includes the above installation system wherein the horizontally disposable supporting member has a reduced cross-section portion adapted to be mounted on and between the projecting elements, whereby shoulder portions of the horizontally disposable supporting member are engageable with opposing end portions of at least one of the pair of projecting members thereby limiting linear displacement thereof. The present description includes the above installation system wherein the horizontally disposable supporting member has a generally polygonal cross-section. The present description includes the above installation system wherein the projecting elements are of uneven length to facilitate the rotation of the horizontally disposable supporting member while mounted. The present description includes the above installation system wherein the probe assembly includes at least a spring-biased element engageable with the one vat internal wall. The present description includes the above installation system wherein the horizontally disposable supporting member has at least one protrusion extending therefrom that is cooperable with at least one of the upstanding projections for limiting linear displacement of the horizontally disposable supporting member.

The present description includes a method of inhibiting degradation of a food treating medium. The method comprises: providing a container for containing a food treating medium and having at least a conductive portion; and, applying electrons by electrically coupling the conductive portion with one or more sources of electrons provided by a capacitive affect for applying electrons from a surface of the conductive portion to a food treating medium in contact with the conductive portion. The present description includes a system of inhibiting degradation of a food treating medium. The system comprises: a container for containing a food treating medium and having at least a conductive portion; and, one or more sources of electrons provided by a capacitive affect for applying electrons from a surface of the conductive portion to a food treating medium in contact with the conductive portion. The present description includes a method of inhibiting degradation of a food treating medium. The method comprises providing a container for containing a food treating medium and having at least a conductive portion; and, providing a source of electrons associated with the conductive portion having a food treating medium in contact with the conductive portion.

The present description includes a method of inhibiting degradation of a food treating medium. The method comprises providing a container for containing a food treating medium and having at least a conductive portion; and, applying electrons by electrically coupling the conductive portion with one or more sources of electrons provided by a capacitive effect for applying electrons from a surface of the conductive portion to a food treating medium in contact with the conductive portion, wherein the one or more sources are separate and apart from a food treating medium contained in the container.

The present description includes a template assembly for use in installing a retrofittable system including one or more probe assemblies suspended from a supporting assembly of the system relative to a food treating system including one or more vats having vat sidewalls for cooking oil for which probe assemblies of an installation system are to be placed in engagement. The template assembly comprises: an elongated supporting device that may be supported in a stationary relationship relative to a food treating system; and an assembly selectively movable to different engagement positions on the elongated supporting device to be placed at one or more positions therealong, the assembly includes a depending element having a construction that generally corresponds to a probe assembly of the system to be installed, the assembly allows the depending element to move to different axial positions along the length of the elongated supporting device and engage sidewalls of a vat, thereby allowing measurement of the position of the depending element relative to the length of the supporting device in response to being in an engaged position with a vat sidewall.

The present description includes having that last noted template assembly wherein the assembly is a slidable assembly that is slidable along the length of the elongated supporting device. The present description includes having that last noted template assembly wherein the depending element is a probe assembly adapted to engage a vat sidewall. The present description includes having that last noted template assembly wherein the depending element is sized and shaped to resemble an actual probe assembly to be used for retrofitting. The present description includes having that last noted template assembly wherein the elongated supporting device includes a plurality of segments that may be joined together in end-to-end relationship to arrive at a length corresponding to a length of a vat system to be retrofitted. The present description includes having that last noted template assembly wherein the segments have markings thereon for facilitating measuring the position of the assembly on the supporting device. The present description includes having that last noted template assembly wherein the depending element is vertically positionable to different positions along a supporting element extending from the elongated supporting device. The present description includes having that last noted template assembly wherein the supporting element includes markings thereon for facilitating measuring the position of the depending element relative thereto. The present description includes having that last noted template assembly wherein the supporting device, assembly and depending element are in a fixed orientation for shipping.

The present description includes a process of retrofitting a food treating system including one or more vats, each of which has vat sidewalls. The process comprises: providing a template assembly that includes an elongated supporting device that may be supported in a stationary relationship relative to a food treating system; and an assembly selectively movable to different engagement positions on the elongated supporting device to be placed at one or more positions therealong, the selectively movable assembly further includes a depending element having a construction that generally corresponds to a probe assembly of a system to be installed; utilizing the selectively movable assembly to allow the depending element to move to different axial positions along the length of the elongated supporting device and engage sidewalls of a vat, thereby allowing measurement of the position of the depending element relative to the length of the supporting device in response to being in an engaged position with a vat sidewall; measuring a length of an elongated supporting member relative to the one or more vats and the position of the depending element relative to the length of the supporting device in response to the device being in an engaged position with a vat sidewall so that such position may be used to locate a probe assembly when a retrofitting system is installed; utilizing a supporting assembly of an installation system including a generally horizontally disposable supporting member having a measured length to be supported by one or more vats; and, utilizing at least one or more generally extending supporting members depending along the length of the horizontally disposable supporting member, wherein each of the one or more extending supporting members is couplable to the horizontally disposable supporting member at one end and is couplable to a probe assembly at another end thereof, the one or more extending supporting members is couplable to the horizontally disposable supporting member at a location corresponding to the position of the depending element relative to the length of the supporting device in response to the supporting device being engaged with a vat sidewall.

The present description includes the last noted process comprising locking the template assembly in a fixed orientation after the one or more depending elements have been added to the elongated supporting device at locations corresponding to desired locations of probe assemblies, whereby the template assembly may be shipped for manufacturing.

The present description includes a method of inhibiting degradation of a food treating medium. The method comprises: providing a container for containing a food treating medium and having at least a conductive portion; and, applying electrons by electrically coupling the conductive portion with one or more sources of electrons provided by a capacitive affect for applying electrons from a surface of the conductive portion to a food treating medium in contact with the conductive portion.

The present description includes a system of inhibiting degradation of a food treating medium. The system comprises: a container for containing a food treating medium and having at least a conductive portion; and, one or more sources of electrons provided by a capacitive affect for applying electrons from a surface of the conductive portion to a food treating medium in contact with the conductive portion.

The present description includes a method of inhibiting degradation of a food treating medium. The method comprises: providing a container for containing a food treating medium and having at least a conductive portion; and, providing a source of electrons associated with the conductive portion having a food treating medium in contact with the conductive portion.

The present description includes a method of inhibiting degradation of a food treating medium. The method comprises: providing a container for containing a food treating medium and having at least a conductive portion; and, applying electrons by electrically coupling the conductive portion with one or more sources of electrons provided by a capacitive effect for applying electrons from a surface of the conductive portion to a food treating medium in contact with the conductive portion, wherein the one or more sources are separate and apart from a food treating medium contained in the container.

The present description includes a system adapted to be retrofitted to a food treating apparatus configured for holding a food treating medium. The system comprises: a monitoring assembly couplable to the food treating apparatus for monitoring at least a quality parameter of a food treating medium to obtain a predetermined value correlated to quality; and a stabilizing assembly couplable to the food treating apparatus and being operable for applying electrons to a food treating medium in response to the predetermined value so as to inhibit degradation of the food treating medium.

The present description includes an apparatus comprising: a support assembly configured for supporting a food item and being removably received within a vessel holding food treating medium; and a low voltage probe attached to and spaced from the support assembly in such a manner that when the support assembly is in the vessel the low voltage probe is at least partially immersible in a food treating medium and a supported food item contacts the food treating medium.

The present description includes a system that comprises: a vessel that includes at least a wall having a portion for defining at least a first chamber and a second chamber divided by a common wall for holding cooking oil; a probe supporting assembly mountable on the common wall for supporting a low voltage probe in each of the first and second chambers so that that the probes are within the cooking oil, wherein the probe supporting assembly supports the probes at a position below a container that is insertable into at least of one the first and second chambers.

The present description includes a method of retrofitting a food container for inhibiting degradation of a food treating medium containable in the food container. The method comprises: providing a container for containing a food treating medium and having at least a conductive portion; and, applying electrons by contacting the conductive portion with one or more sources of electrons provided by a capacitive affect for applying electrons from a surface of the conductive portion to a food treating medium in contact with the conductive portion.

An aspect of the present description is a method, apparatus, and system for inhibiting degradation of a food treating medium.

An aspect of the present description is a method, apparatus, and system for inhibiting degradation of a food treating medium by applying electrons to a vessel containing the food treating medium.

An aspect of the present description is a method, apparatus, and system for inhibiting degradation of a food treating medium by applying electrons by a capacitive effect to a vessel containing the food treating medium.

An aspect of the present description is a method, apparatus and system for extending the food treating media by applying electrons to a vessel containing the food treating medium, whereby the source of electrons is not in contact with the food treating medium.

An aspect of the present description is a method, apparatus and system for extending the food treating media by applying electrons by a capacitive effect to a vessel containing the food treating medium, whereby the source of electrons is not in contact with the food treating medium.

An aspect of the present description is a method, apparatus, and system for extending media quality based on inhibiting its degradation by steadily or dynamically applying electrons.

An aspect of the present description is a method, apparatus, and system for monitoring at least a quality parameter of a food treating medium to obtain a predetermined value correlated to quality; and applying electrons to the food treating medium so as to inhibit degradation of the food treating medium.

An aspect of the present description is a method, apparatus, and system to provide an extremely quick and inexpensive approach for retrofitting existing food treating systems.

An aspect of the present description is a method, apparatus, and system to provide an extremely quick and inexpensive approach for saving considerable amounts of food treating media overtime by extending the life of the food treating media.

An aspect of the present description is a method, apparatus, and system for dynamically applying electrons to the food treating medium in order to alter degradation of the food treating medium.

Still another aspect of the present description is a method, apparatus, and system that achieve the foregoing in a manner that increases significantly the useful life of cooking media.

A further aspect of the present description is a method and system for supporting and controlling a plurality of probe assemblies in one or more vats.

Aspects of the present description include a system and method of installing the same enable versatile, low cost, and yet easy retrofitting of a variety of different shaped and sized fryer systems or the like which utilizes multiple probe assemblies.

Aspects of the present description include a system and method that facilitate the cleaning of fryer vats as well as the probe assemblies themselves.

Aspects of the present description include a system and method which provides for a template assembly that allows easy retrofitting of a vat system, wherein the template system is a mock-up or replica of the installation system to be actually used in retrofitted.

Aspects of the present description include a system and method that provides for a template assembly that lock the template assembly in a fixed orientation, whereby the template assembly may be shipped for manufacturing.

The aspects described herein are merely a few of the several that can be achieved by using the present description. The foregoing descriptions thereof do not suggest that the present description must only be utilized in a specific manner to attain the foregoing aspects.

These and other features and aspects of the present description will be more fully understood from the following detailed description of exemplary embodiments. It should be understood that the foregoing generalized descriptions and the following detailed descriptions are exemplary and are not restrictive of the present description.

### BRIEF DESCRIPTION OF THE DRAWINGS

FIG. 1 is a schematic perspective view of one aspect of a food treating system made according to one exemplary embodiment of the present description for suppressing oxidation and hydrolysis of a food treating medium.

FIG. 2 is an enlarged schematic view of a portion of the food treating system illustrated in FIG. 1 illustrating placement of a monitoring system that may be easily retrofitted to the system.

FIG. 3 is a perspective view of one optical monitoring system useful in the food treating system of the present description.

FIG. 4 is a perspective view of another exemplary embodiment of optical monitoring system that may be utilized.

FIG. 5 is a perspective view of a low voltage probe useful in the food treating system of the present description.

FIG. 6 is block diagram of a food treating method that may be implemented by the food treating system of the food treating system.

FIG. 7 is a bar chart indicating improved results of reducing hydrolysis of oil according to the present description.

FIG. 8 is a bar chart indicating improved results of slowing oxidation according to the present description.

FIG. 9 is a schematic view of a fryer including a plurality of vats and low voltage probes for use therewith.

FIG. 10 is a schematic view of a fryer including a plurality of fryer baskets having low voltage probes connected thereto.

FIG. 11 is a schematic view of a fryer similar to FIG. 10 including a plurality of fryer baskets having low voltage probes connected to the fryer vat.

FIG. 12 is a schematic view of a low voltage probe connected to a fryer vat walls.

FIGS. 13A and 13B are plots indicating improved results of reducing free fatty acids relative to time.

FIGS. 14A and 14B are bar charts indicating improved results of reducing TPC values relative to time.

FIG. 15 illustrates another exemplary embodiment of the present description.

FIG. 16 illustrates another exemplary embodiment of the present description.

FIG. 17 illustrates another exemplary embodiment of the present description.

FIG. 18 illustrates an enlarged schematic view of a portion of a bendable and shape retentive tube holding a probe.

FIG. 19 illustrates a schematic view of a system for positioning each of a plurality of probes into corresponding separate vats of a fryer.

FIG. 20 illustrates an exemplary embodiment of a system retrofitted to a vat system according to the present description.

FIG. 21 illustrates an exemplary embodiment of a template assembly according the present description.

FIG. 22 illustrates an elevation view of another exemplary embodiment of a probe assembly arrangement according to the present description.

FIG. 23 illustrates an end of view of the probe assembly of FIG. 22.

FIG. 24 illustrates an exemplary embodiment of a retaining assembly according to the present description.

FIG. 25 illustrates another exemplary embodiment of a supporting arrangement of the system illustrated in FIG. 20.

FIG. 26 illustrates an end view of another exemplary embodiment of a probe assembly according to the present description.

FIG. 27 illustrates an elevation view of another exemplary embodiment of a probe assembly according to the present description.

FIG. 28 illustrates an elevation view of another exemplary embodiment of a probe assembly according to the present description.

FIG. 29 is a perspective view of an installation system according to another exemplary embodiment of the present description.

FIG. 30 is a partially segmented elevation view of a horizontally disposable supporting member usable in the installation system.

FIG. 31 is a fragmented and cross-sectional view of the horizontally disposable supporting member mounted on a bracket member mounted on a partition wall defining a part of a vat of a food treating system.

FIG. 32 is a view similar to FIG. 31, but illustrating a fragmented side view of the horizontally disposable supporting member mounted on the bracket member.

FIG. 33 is a view of another exemplary embodiment of a spring-clip for surrounding a probe assembly.

FIG. 34 is a schematic view of the horizontally disposable supporting member rotated to engage an internal vat wall.

FIG. 35 is another exemplary embodiment of a bracket member for holding a horizontally disposable supporting member.

FIG. 36 is a side view of the exemplary embodiment of the bracket illustrated in FIG. 35 shown holding a horizontally disposable supporting member.

### DETAILED DESCRIPTION

According to the present description, provisions are made to improve upon the above noted drawbacks and shortcomings by stabilizing the useful life of food treating media, such as cooking media. Stabilization may be achieved by approaches that effectively, economically extend media quality based on inhibiting its degradation as, for example, by oxidation and hydrolysis through application of electrons on a fixed or dynamic basis. The stabilization may be in response to monitored values related to cooking medium quality, particularly during food treating. Also, provisions are made to do the above in manners that can be effectively and economically retrofitted to existing food treating systems, such as fryers. The words “a,” “an,” and “the” are used interchangeably with “at least one” to mean one or more of the elements being described. By using words of orientation, such as “top,” “bottom,” “overlying,” “front,” and “back” and the like for the location of various elements in the disclosed articles, we refer to the relative position of an element with respect to a horizontally-disposed body portion. We do not intend that the disclosed articles should have any particular orientation in space during or after their manufacture.

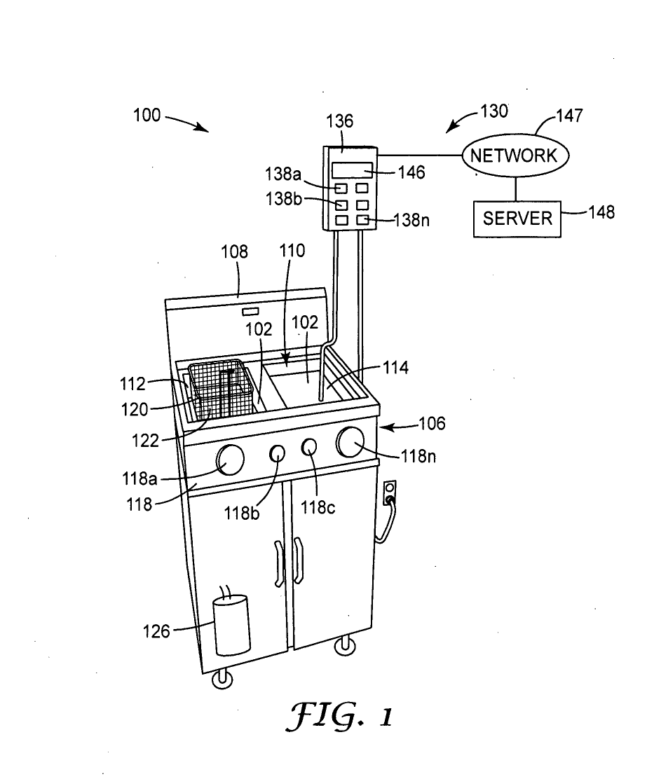

Reference is made to FIGS. 1-14B for illustrating several exemplary embodiments of methods, systems, and apparatus for economically, automatically, steadily, or dynamically, and efficiently stabilizing the quality of food treating media in an on-line and real-time basis. Accordingly, there is a suppression of the rate at which food is subjected to unhealthy or excessive levels of volatile and nonvolatile decomposition products. The volatile and nonvolatile decomposition products may be produced through oxidation and hydrolysis.

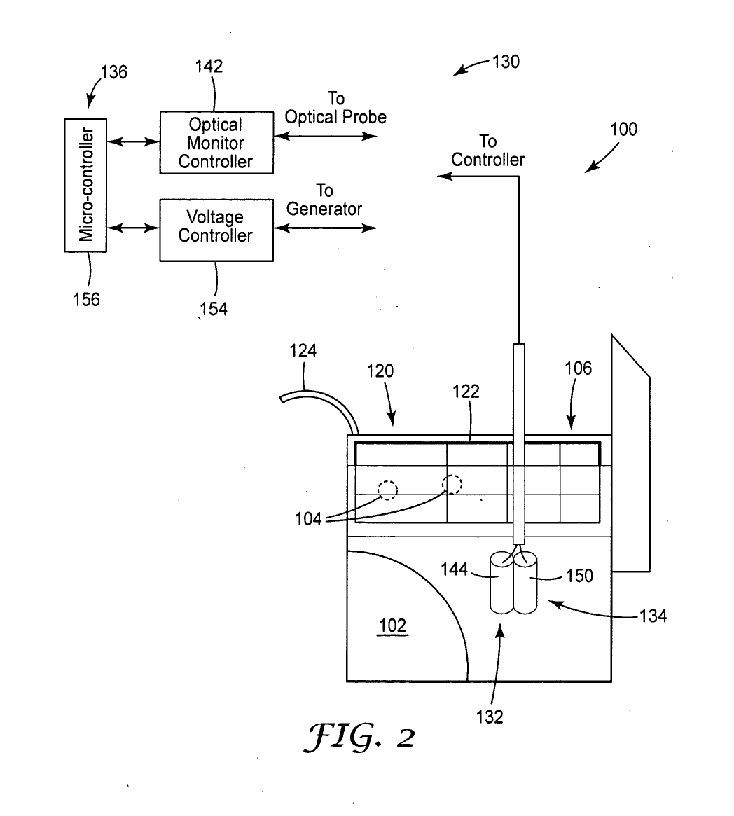

Reference is made to FIGS. 1-8 for illustrating one exemplary embodiment of a food treating system 100 that is operable for enhancing the quality of food treating media 102, such as a cooking medium 102 useful for treating food 104 (FIG. 2) by a suitable food treating process. The food treating media 102 contemplated to have its food treating qualities stabilized by the present description may be oxidizable and hydrolyzable. Such food treating media 102 may include, but are not limited to, cooking oils, fats, water, sauce, or other suitable media. The cooking oils and fats may be vegetable based, animal based, synthetic, or blends thereof and are generally considered to be edible. Examples of vegetable cooking oils that are both oxidizable and hydrolyzable include, but are not limited to, corn oil, soybean oil, canola oil, safflower oil, olive oil, palm oil, rapeseed oil, sunflower seed oil, and cottonseed oil. The food treating methods contemplated include, but are not limited to cooking, frying, heating, roasting, boiling, warming, cooling, steaming, basting, skewering, sauteing, frying, baking, deep frying, steaming or other cooking, storing, cooling, and preparing process.

While the food treating system 100 of the present description includes a fryer 106, it will be understood that the food treating process of the present description may be performed in combination with other vessels, such as, but not limited to, storage containers, cooling containers, preparing containers, warming containers, including without limitation pots, pans, cookware, skillets, kettles, dishes, bowls, woks, appliances, frying baskets or the like.

The fryer 106 illustrated in FIG. 1 may be a known type of fryer that includes a housing assembly 108 including a blower. The housing assembly 108 is adapted to be easily retrofitted in accordance with the present description. The fryer 106 includes a vat 110 comprised of two chambers 112, 114, each of which can hold the cooking medium 102, such as an edible chicken cooking oil 102. In the exemplary embodiment, the chicken cooking oil 102 is a vegetable oil, such as blend of soybean oil and canola oil. Such cooking oil 102 is of the type in which oxidation and hydrolysis may be suppressed by providing electrons thereto as will be explained. Clearly, other types of similar cooking media may be used.

The cooking oil 102 may be heated by the fryer 106 by known mechanisms (not shown) to temperatures that may be in a range of about 325° F. to about 400° F. at which the cooking oil acts to deep fry the food 104 (chicken nuggets, French fries or the like) in a typical manner. A control panel 118 having control knobs 118a-n is provided for controlling the food treating process in a known manner.

Reference is made to FIG. 2 along with FIG. 1 for illustrating a fryer basket 120 of any suitable size and shape that is immersed in the cooking 102 oil held in one the chambers. Typically, the fryer basket 120 includes a generally rectangular food container portion 122 and a handle 124. The container portion 122 may be made as an open wire mesh (e.g., stainless steel) in which the food 104 is supported while being immersed in the cooking oil 102 during food treating. Besides a fryer basket 120, the food supporting assemblies, devices or mechanisms of the present disclosure are intended for supporting food to be treated in a cooking medium. For example, other supporting mechanisms or devices may include, but are not limited to, skewer, pot, griddle, and other similar devices. The fryer 106 may include a pumping system 126 (see FIG. 1) that is able to circulate the cooking media through a filter system (not shown) and/or replenish the vat 110 with additional new cooking media from a source (not shown) as is generally done after a day of deep frying.

In accordance with the present description, the fryer 106 may include a monitoring and stabilizing system 130 that may be easily added or retrofitted to the fryer 106. Alternatively, the present description envisions that such a monitoring and stabilizing system 130 may be integrated in a fryer as well. Alternatively, just the stabilizing system may be used as will be described herein. The monitoring and stabilizing system 130 of the present exemplary embodiment is operable for either continuous or intermittently operation. The present embodiment may monitor the quality of the cooking medium 102, as well as automatically stabilize it based on the monitored results. In particular, the monitoring and stabilizing system 130 may include a monitoring assembly or device 132 and a stabilization assembly or unit 134 that are controllable by a controller 136 that includes a plurality of control buttons 138a-n (collectively 138) as will be explained. A wide variety of systems are contemplated for carrying out the monitoring and stabilization.

In regard to the monitoring functions, the present description envisions monitoring at least a quality parameter of the cooking oil 102 to obtain a predetermined value correlated to its quality, as will be explained. Thereafter, manually or automatically, the cooking oil 102 may be enhanced by applying electrons thereto in response to obtaining a predetermined monitored value. Accordingly, oxidation and hydrolysis of the cooking oil are inhibited and its rate of degradation suppressed. As a result, increases of decomposition products, for example free fatty acids, TPC, and the like, that diminish cooking media quality, are suppressed. There are a number of physical (e.g., viscosity, density, smoke, etc.) and chemical (free fatty acids, total polar components, etc.) quality parameters associated with the quality of food treating media. The present description envisions monitoring one or more of a vast number of quality parameters. For example, parameter values may be monitored by taking measurements that include, but are not limited to luminescent measurements, dielectric constant measurements, visible and infrared spectroscopy measurements, Fourier transform infrared (FTIR) measurements, column chromatography measurements, temperature measurements, viscosity measurements, density measurements, e-nose measurements, and ultrasonic measurements. Further examples of monitoring quality parameters include the approaches described in commonly assigned and copending U.S. Patent Applications: 61/033,487 entitled “METHODS AND DEVICES FOR MONITORING FRYING OIL QUALITY” filed Mar. 4, 2008; and 61/033,481 entitled “MONITORING OF FRYING OIL USING COMBINED OPTICAL INTERROGATION METHODS AND DEVICES” filed Mar. 4, 2008, which are incorporated herein and made a part hereof.

In one of the exemplary embodiments of the monitoring device 132, quality is measured by monitoring a luminescent value derivable from measuring a fluorescent level signal emitted by the cooking oil 102 in response to the presence of decomposition products. One such method that may be used occurs when the cooking oil is subjected to an irradiating process similar to that described in commonly assigned and copending U.S. Patent application No. 61/007,894 entitled “DEVICE FOR THE QUALIFICATION OF COOKING OILS, AND METHODS” filed on Jan. 8, 2007. Such application is incorporated herein and made a part hereof. Only those portions thereof believed necessary for a description of the present disclosure are described.

The monitoring device 132 may be configured to determine the quality of cooking oil (e.g., frying oil) in an easy and real-time manner. The monitoring device 132 measures the fluorescent levels of the cooking oil, which may correlate to the levels of TPC in the cooking oil, and then compares the fluorescent signals generated to values of a predetermined curve or table associated with various qualities of the cooking oil in memory of the monitoring device as is known. In FIG. 2 there is illustrated an embodiment of the monitoring device 132. The monitoring device 132 may include an optical monitor controller or meter 142 and an optical probe 144 which is operatively engageable with the meter 142. It will be understood that a wide variety of other fluorescent monitoring systems including meters and probes may be used. One example of such a probe is commercially available from Ocean Engineering Corporation. Also, the monitor probe 144 may be immersed in the oil constantly or intermittently

The optical probe 144 monitors the luminescent response of the cooking medium and is operably joined to the meter 142 without having to remove a sample from the cooking oil batch in the vat. The optical probe 144, under control of the meter 142, irradiates the cooking media 102 and the meter measures the fluorescent response through the optical probe 144. The probe 144 may include an optical fiber for transmitting the irradiation beam to the cooking oil and one for transmitting the fluorescent response to the meter 142. The meter 142 forms a part of the controller 136. The meter 142 may include known features, such as the buttons 138 for inputting information (e.g., the composition of the oil), appropriate mechanisms, such as lasers or LED's to provide the irradiating energy and appropriate mechanism, such as a photodetector, to measure any fluorescent response, as well as electronics that compare the measured level of response to a predetermined curve or table of values. A display 146 (FIG. 1) may be provided for the user to read the results. Of course, the present description contemplates other suitable devices for inputting and outputting data. As another example, the display may include a green light to indicate the oil sample is still acceptable and a red light to indicate the oil should no longer be used. Yellow and/or orange lights may be present between the green light and red light to indicate a progression. The display may be a quantitative display, providing a specific number of, e.g., TPC, free fatty acids in the oil.

Many different approaches may be used to integrate the operation of the monitoring with the stabilizing. The controller 136 of the present exemplary embodiment may control both the optical monitor controller or meter 142 and the low voltage controller 154. In this embodiment, the controller 136 may include a microcontroller 156 that operates and controls both the meter 142 and the low voltage controller 154 in a manner that is described below. The microcontroller may in response to signals from the meter control operation of the stabilizing unit. For example, the stabilizing unit may provide fixed or varied outputs (e.g., pulsed). In the exemplary embodiment, the microcontroller may include a programmable electronic system, such as a microprocessor, programmable logic device, portable computer system or the like. The programmable electronic device may be programmed by a module or mechanism (not shown) which is an application that allows the monitoring assembly and the stabilization unit to function as described herein. Alternatively or additionally, both the meter 142 and the controller 154 may be connected by a network 147 to a programmable electronic system, such as a server system 148. The server system 148 may also control a printer (not shown) for generating reports and may control the stabilization unit as will be described. A database within a memory of a microprocessor may hold the stored information regarding the values of the spectral fluorescent response and corresponding information relating to cooking oil quality. It will be appreciated that the functions of the meter and the ion generator control may be combined into a single control unit instead of being comprised of separate control devices of each being operatively coupled through the microcontroller.

Other parameter monitoring units may be used that need not be operatively coupled to a stabilization unit, but which can provide values for determining whether a stabilizing unit may be operated and at what values and/or for how long. For example, reference is made to FIG. 3 for illustrating another exemplary embodiment of a monitoring device 332 for measuring the fluorescent response of the cooking medium. This embodiment may be similar to the embodiment described in the last noted patent application. Accordingly, only those details thereof that are believed pertinent will be described herein. The monitoring device 332 accepts a sample of cooking oil that would be placed in the sample receiver 344, as for example, by a swab, tube or pipette at least partially receivable within the sample receiver 344. An additive, such as a fluorescent marker, may be present within receiver 344 or may be added after the oil sample. The receiver 344 may be inserted into or against the meter 322 that irradiates the cooking sample and measures the luminescent response. The meter 322 may include features noted above of the last embodiment. A user may operate the stabilization unit or other control devices may automatically operate the stabilization unit.

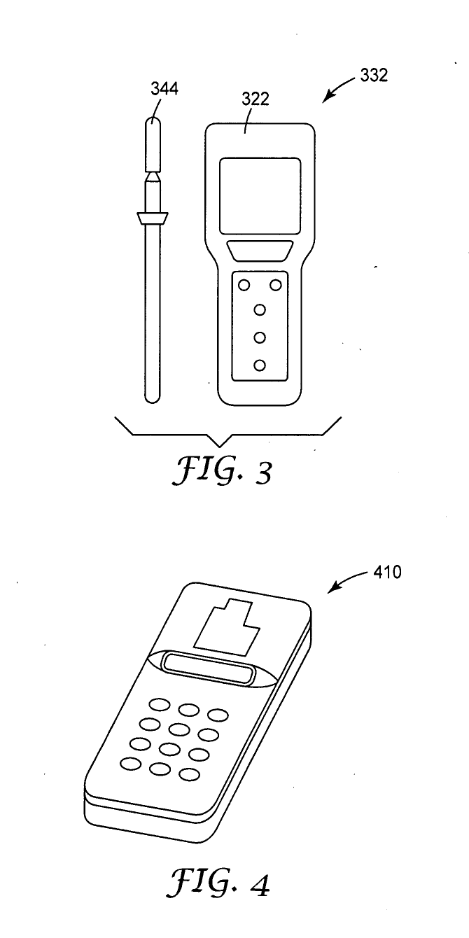

Another exemplary embodiment of monitoring may be done by a hand-held device 410 depicted in FIG. 4. This embodiment as the preceding one need not be operatively connected to a stabilizing unit, but may provide independent information regarding oil quality. The hand-held device 410 may be similar to that described in the last noted patent application. The hand-held device is suitable for measuring the emitted fluorescent signals of the cooking oil. In this embodiment, the device 410 is a non-contact, optical sensor, configured for irradiating the oil sample and measuring the fluorescence without contacting the oil. The device 410 includes features, such as those described above for inputting information (e.g., the composition of the oil), appropriate means to provide radiation and appropriate means to measure the fluorescent response, electronics that compare the measured level to a threshold, and a display for the user to read the results. A database within a memory or microprocessor in device 410 may hold the stored information regarding the values of the spectral fluorescent response and its corresponding information relating to cooking oil quality. A suitable source of power may be provided to operate the device 410. A user may operate the stabilization unit or other control devices may automatically operate the stabilization unit.

In several of the above described embodiments, the spectral frequencies used for monitoring may be visible light. Visible light having a wavelength of 470 nm is one exemplary wavelength for irradiating the oil to be tested, particularly if no fluorescent markers are used. The meter 142 then measures the fluorescence level, at a wavelength different than the irradiating wavelength. If wavelengths of 470 nm are used for irradiating, a measuring wavelength may be 520 nm. Different spectral frequencies may be used including those for eliminating opportunity for back scatter and background noise. Accordingly, the optical monitoring devices of the present disclosure are configured to determine the quality of cooking oil (e.g., frying oil) in an easy and real-time manner.

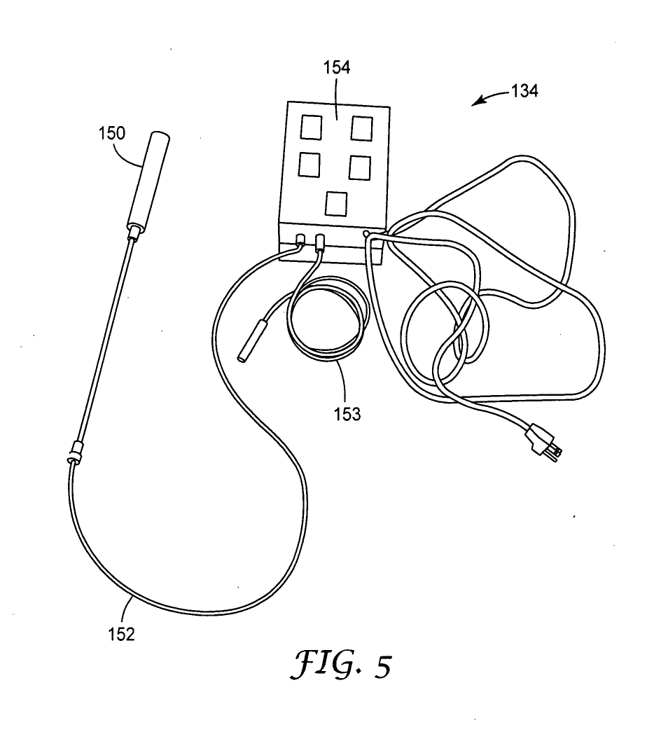

Reference is made to FIG. 5 along with FIGS. 1 and 2 for describing one exemplary embodiment of a stabilization assembly or unit 134. The stabilization assembly 134 may be a low output voltage source, such as a so-called negative ion generator described in Japanese Patent Document Granted Patent Publication (B2) (11) U.S. Pat. No. 3,463,660 (P3463660)45) Date Issued: Nov. 5, 2003, or the like. The stabilization assembly 134 is operable in response to a voltage being applied thereto for acting as a source of electrons that will be applied to cooking medium as will be described. A wide variety of suitable approaches may be used to provide a low output voltage source, including but not limited to a capacitive effect, an electric field, or the like. As such, the stabilization assembly 134 contemplated for supplying and applying electrons provides a highly versatile source that may be used in any location, including submerged in the cooking oil or not submerged in the cooking oil. In the present illustrated embodiments, a probe may contact a conductive portion of the vat or be an integral part of a vat as described herein. Also, since a capacitive effect may be used, the flow of electrons may be controlled and varied easily. It will be appreciated that the foregoing described versatility is not necessarily easily and cheaply available should a circuit be formed as an integral part of a vat for the cooking oil. The capacitive effect of the foregoing probe enhances the versatility of the present description to be used in retrofitting existing fryers and the like, wherein the probe need not be placed in a cooking oil, the food need not be present in the cooking oil, and the cooking vessel need not form part of a circuit for applying electrons. In one exemplary embodiment, the stabilization unit 134 may include a low voltage probe 150. The 150 may include an electrode surrounded by an insulator material that is in turn surrounded by a metal shield. As understood, a capacitive effect may generate electrons that may be transferred to the cooking oil by the metal shield. In one exemplary embodiment, the probe 150 may be of the type that is commercially available from Rejuvenoil, Hoei America, Inc., Buffalo Grove, Ill. In such an approach, there is provided a semiconductor element that in response to a voltage supplied thereto supplies a low voltage. The low voltage in turn is transferred to a conductive shield or housing that surrounds the semiconductor element and transfers the electrons generated. Also, such probe may be described in Japanese Patent Document Granted Patent Publication (B2) (11) U.S. Pat. No. 3,463,660 (P3463660)45) Date Issued: Nov. 5, 2003. In the latter approach, the semiconductor material is exposed. The semiconductor materials in both of the foregoing exemplary units are described in the foregoing Japanese Patent document which description thereof as well as other equivalent structures are incorporated herein as a part hereof. It will be appreciated that the semiconductor materials are not particularly limited thereby. It will be understood, the other equivalent materials may be used consistent with the present description. The capacitive effect generated by the foregoing described devices enables generating electrons in a manner that avoids shock. It will be understood that a wide variety of capacitor devices, with or without semiconductor materials, may be used to provide the amount of electrons considered sufficient for inhibiting oxidation and reduction as described in the present description. In this regard, whatever capacitors including whatever materials are used and whatever voltage or voltage amounts that are applied, a sufficient amount of low voltage is generated at a rate to provide for the degree of inhibition of oxidation and reduction contemplated by the present description. A tubing 152 or sheath covers a cable 153 to the probe from a control box 154 that functions as the meter and may be located in the controller 136 (FIG. 2) and is under control of the microcontroller unit 156. The control box 154 may be powered by the same source as for the controller and the fryer. The present description contemplates that other sources of electrons may be applied to the cooking medium. While the present embodiment discloses one such low voltage probe, the present description contemplates more than one that may be operated so as to increase the surface area of the probes that emit low voltage into a cooking medium. The latter may be placed in contact with a conductive surface of the vat and as a result the surface area of the vat may then transmit the electrons to the cooking medium. The probe does not have to be in the oil. As will be explained, the greater surface area enhances distribution of the electrons and hence enhances inhibition of the oxidation and hydrolysis.

The controller of the present description may, in response to the monitored signal from the monitor device activate the stabilization unit. The probe emits the electrons at a fixed rate considered safe for the cooking medium and sufficient for inhibiting oxidation and hydrolysis. Typically, probe 150 is operated at about −12 volts DC. The present description envisions that the emission rate may be varied. For example, with the low voltage probe 150 contacting the vat wall, its rate of distribution may vary in response to the voltage applied thereto. For example, the voltage may vary from about −0.1 v to −12 v. or a value that is less than the cooking oil or cooking medium would break down. Accordingly, the present description envisions that the amount of electrons can be varied depending on the desired results, such as how quickly the oxidation and hydrolysis are to be slowed, the cooking medium, the food being treated or any combination of the above. The output of the stabilization unit varies based on the monitored value reached by the monitoring device. The present description contemplates other approaches for enhancing useful life of the cooking oil. It will be understood by those of skill in the art that the term “stabilization” refers to extending the life of the food treating media.

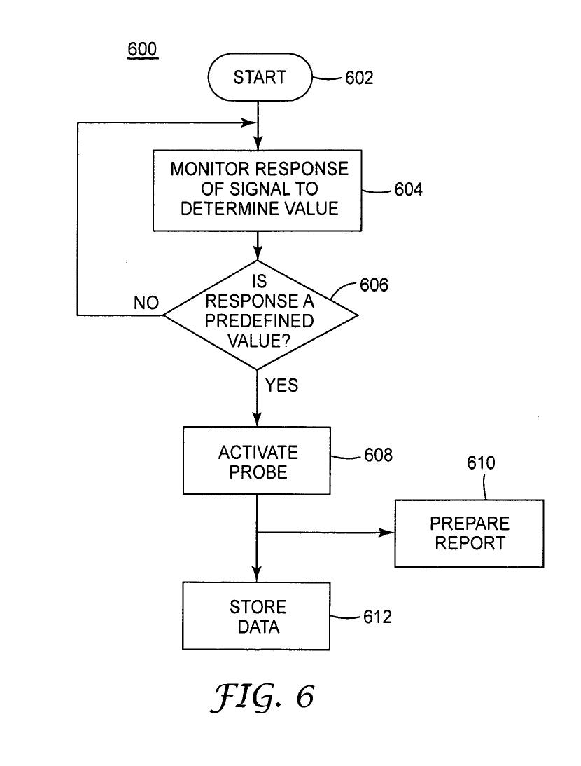

FIG. 6 is flow diagram of one exemplary embodiment of a monitoring and stabilization process 600 of the present description that may be carried out by the food treating system 100. In this regard, the process 600 may commence in a Start block 602 in response to a user activating a start button of the controller 136. According to the process 600, monitoring of the cooking medium 102 is commenced. In a Monitor Response of Signal to Determine Value block 604, the monitoring of the cooking oil quality may be commenced by emitting a timed laser triggering signal through the optical probe 144 (FIG. 2). The laser triggering signal causes a wavelength (e.g., 470 nm) to be emitted from the probe. The optical probe 144 collects the fluorescent response that is returned to the optical monitor control or meter 142. In the Monitor Response of Signal to Determine Value block 604, the photodetector of the meter 142 provides a value of a response to the triggering signal as noted above. In Is Response a Predetermined Value? block 606, the meter 142 determines whether the value of the signal responding to the triggering signal is of a predetermined value that is indicative of poor or marginal quality oil for which stabilization is desired. Such a determination is based on a comparison of the received value with the stored spectral values indicative of poor or marginal quality oil. If the decision is No, that the quality is not the predetermined value corresponding to one of the values indicative of poor or marginal quality and is otherwise good, then the process 600 returns to block 604 for continuing the monitoring. If the decision is Yes, that value is a predetermined value, indicating that the oil is of poor or marginal quality, then the process 600 proceeds to the activate the stabilizing unit in an Activate Probe block 608. In this regard, the predefined signal activates the microcontroller 156, which, as noted, is programmed to operate the stabilization unit. Accordingly, in block 608 the low voltage probe becomes operable to discharge electrons in the vat. As a result, the oxidation and hydrolysis are inhibited. Thereafter, the process 600 may store such data in Store Data block 612 either through a memory of the microcontroller or the server 148. The present description envisions that the process may generate reports in Prepare Report block 610. The present description is not limited to the foregoing functions being carried on, but envisions a wide variety of functions may be added and altered, such as issuing reports during other aspects of the process 600.

### EXAMPLES 1 and 2

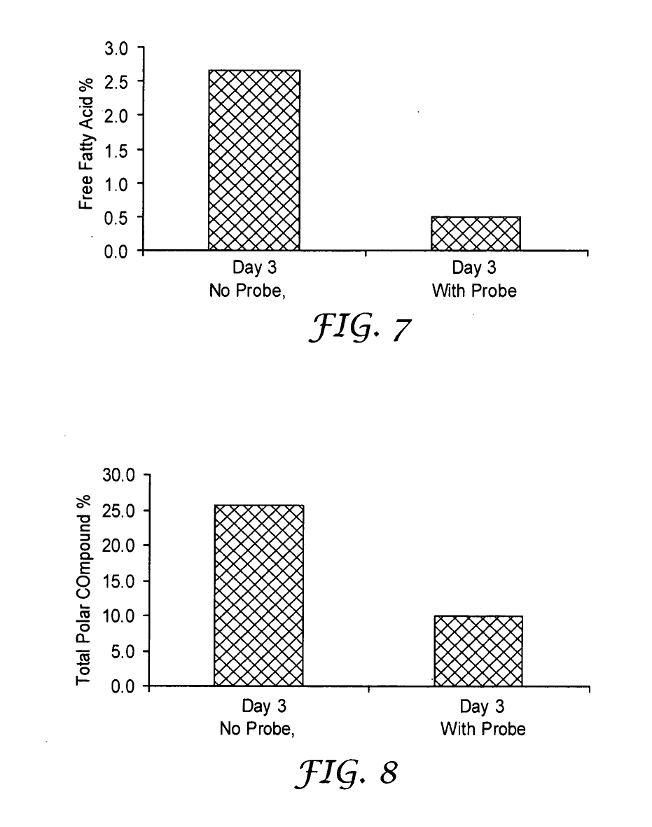

FIGS. 7 and 8 depict bar charts respectively illustrating the advantageous results achievable as a result of the stabilization process of the present description, wherein an ion generator probe 150, such as Model No. RA-L2 that is commercially available from Rejuvenoil Hoei America, Inc. was utilized in about 52 liters of trans-fat free vegetable blend oil for the deep frying of chicken nuggets. For conducting the comparison substantially the same testing conditions were used. The weight percentage of free fatty acids (FFA %) were measured using a known testing process AOCS Official Method Ca 5a-40. The percentage of Total Polar Compounds (TPC %) were determined with 3M commercial product, 3MTM PCT 120. A cooking oil, such as a blend of corn oil and soy oil was used and is commercially available and was used throughout three (3) days. The cooking oil was topped to 52 litters at the end of days so that at the start of each testing day, the same amount cooking oil was available for deep frying day. Replenishment was approximately comparable for the samples having no probe and the sample having the low voltage probe. In terms of volume of cooking oil, replenishment oil was considered to be a negligible factor. During deep frying operations, the cooking medium was heated to a temperature in a range of about 325° F. to about 375° F. At the end of each cooking day, the cooking medium was drained from a vat and filtered through a filtration system. The deep fryer was a commercially available deep fryer, such as one commercially available from Henny Penny, Eaton, Ohio; Pitco Frialator, Inc. Concord, N.H.; or Frymaster, Shreveport, La.

It is observable that after three (3) days of comparison testing there was a significant reduction in the amount of free fatty acids (FIG. 7) and total polar components (FIG. 8) in the cooking medium when using the low voltage probe as compared to non-use of such a probe. After three days, the deep fried chicken nugget batch without the low voltage probe test indicated that the free fatty acid (FFA %) and the total polar components (TPC %) were at levels of about 2.70 percent by weight of the batch and 25.7 percent by weight of the batch; respectively. These levels may exceed the predetermined levels indicative of oil replacement. As noted, these predetermined levels may be related to private or government standards. It will be appreciated that values in excess of the noted predetermined levels may suggest that the cooking medium should be replaced because the levels of the free fatty acids and TPC are considered to be unacceptable.

In contrast, after three (3) days with the low voltage probe 150, the free fatty acid (FFA %) level of the chicken oil batch is at about 0.50%, while the (TPC %) level is at about 10.0%. Both values are below the predetermined values associated with degraded oil. Since these values remain below the replacement levels, essentially the same batch of cooking oil may continue to be used. Advantageously, this results in significant cost savings that are derivable from extending useful life of the cooking medium. Moreover, significantly more chicken nuggets were able to be processed when the probe was used. In fact, about twice as many chicken nuggets by weight were able to be cooked using one probe as opposed to no probe.

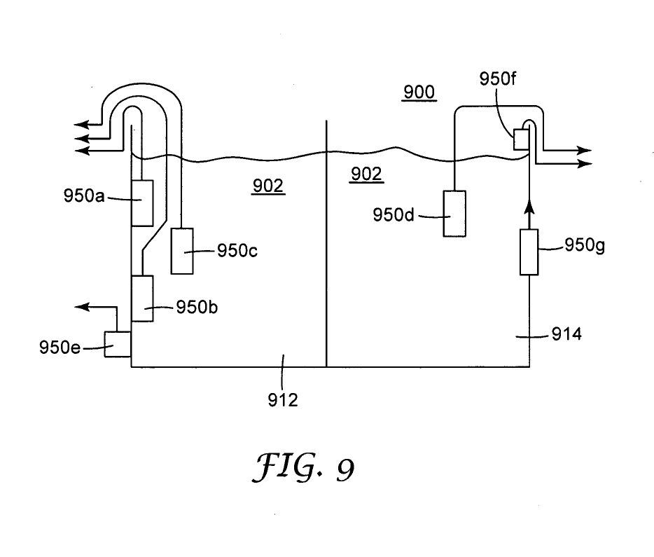

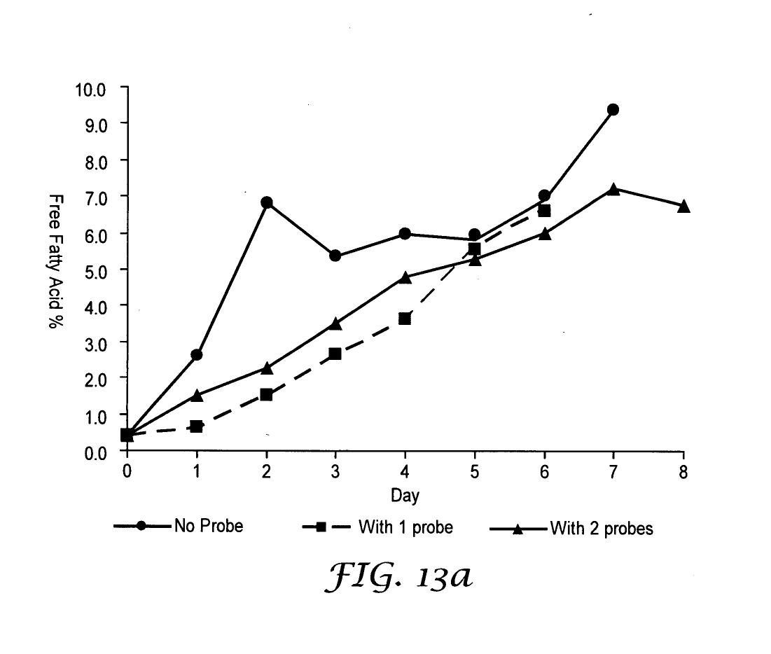

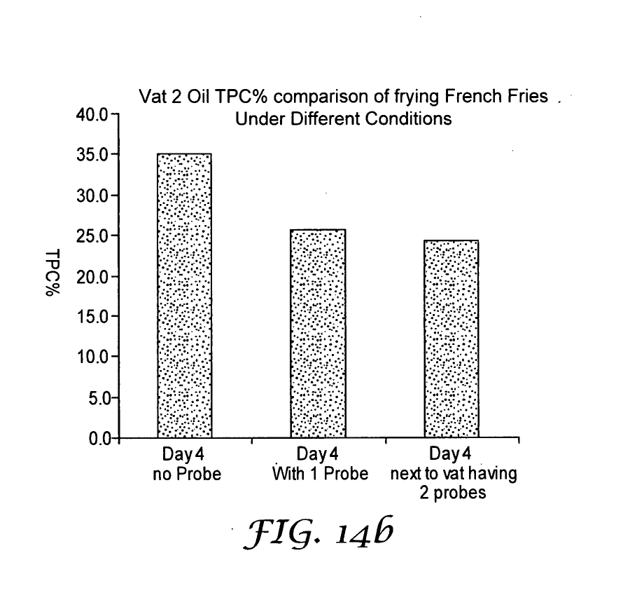

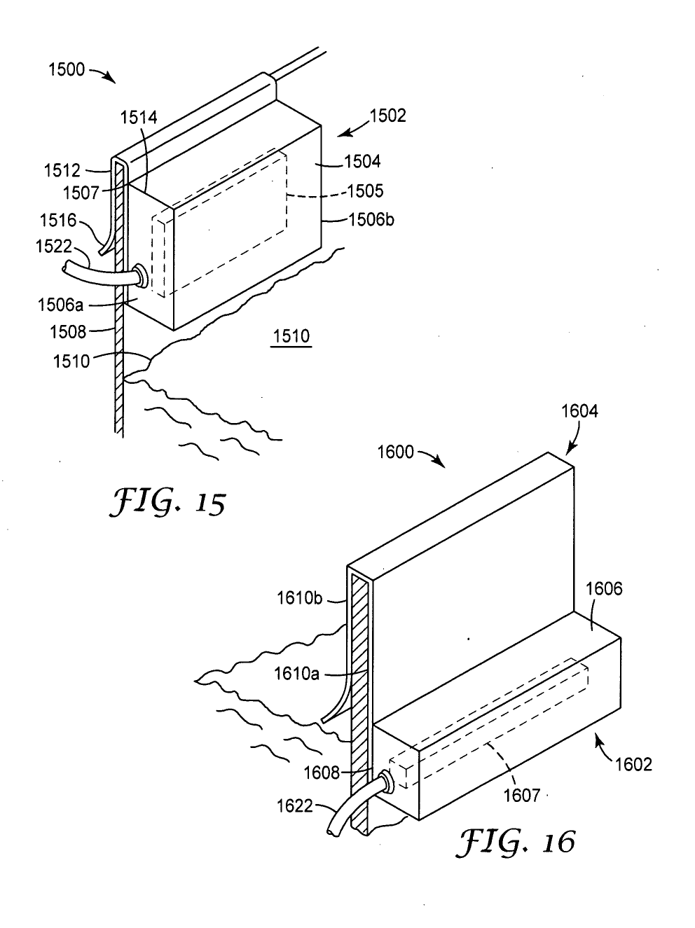

FIG. 9 is a schematic version of another system 900 using an probes 950a,b being attached to a conductive wall of one the two vat chambers 912 (Vat 1) and 914 (Vat 2) of a fryer. The probe 950c may be suspended in vat chamber 912 (Vat 1) alone while probe 950d may be suspended in vat chamber 914 (Vat 2). The advantages of the foregoing will be described in the context of Table 1 below along with FIGS. 13A and 13B. By having the probes 950a,b attached as noted, the electrons flow to the interior surface of the two vat chambers 912 Vat 1 and 914 Vat 2. The probe 950e may be connected to the exterior wall of one of the vat chambers and the electrons will flow conductively through conductive portions of the system to the cooking oil 902. It will be appreciated that the benefits of the present description may be achieved by the probe contacting the outside of the conductive fryer vat without being immersed in cooking oil. It will also be appreciated that the probes may be attached conductively to the interior conductive portions of the fryer as well as being immersed in the cooking oil. As will be pointed out, the present description describes extending useful life even with the probe on the outside of the wall of just one of the vats. In this regard, a probe 950f is connected to an interior wall surface of the vat so that it is completely out of the cooking oil. The probe 950f may have a configuration that includes a coupling assembly that enables the probe to be coupled to and supported in intimate engagement with and by a common vat wall. The probe 950f may also have the configuration similar to the one described the exemplary embodiment in FIGS. 15 and 16 supra, whereby it is placed in intimate engagement with the vat wall for transferring electrons thereto. The system of the present description comprises another probe 950g that is built-in a wall of the vat so that one of its surfaces may be in intimate and direct engagement with cooking oil. It is also pointed out that the source of power for the probe 950g may be internal of the vat wall or external to it. It will be appreciated that the supplied power and the area of the surface engaging the cooking oil may be selected to provide for an electron rate that achieves the oxidation and reduction that are described herein and this power supply is also built-in to the vat wall.

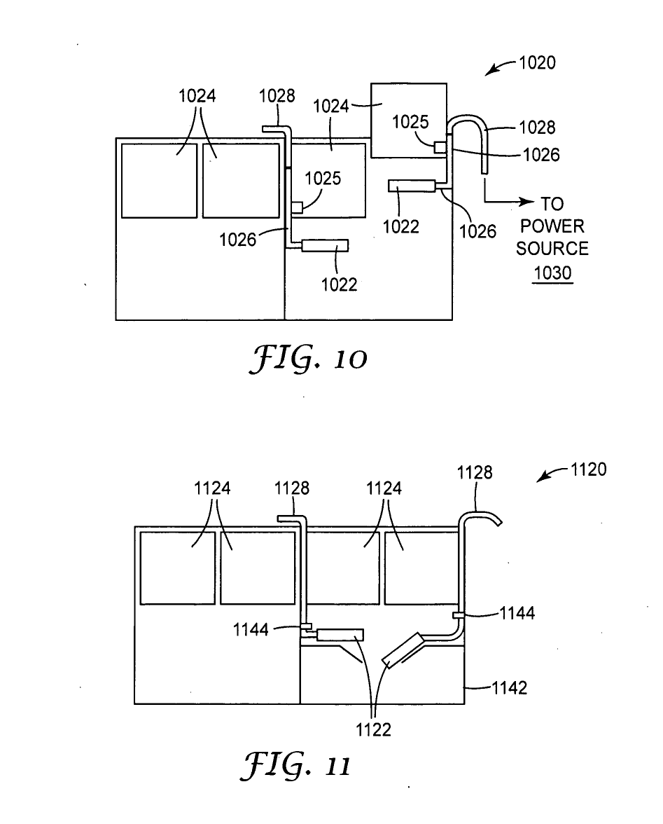

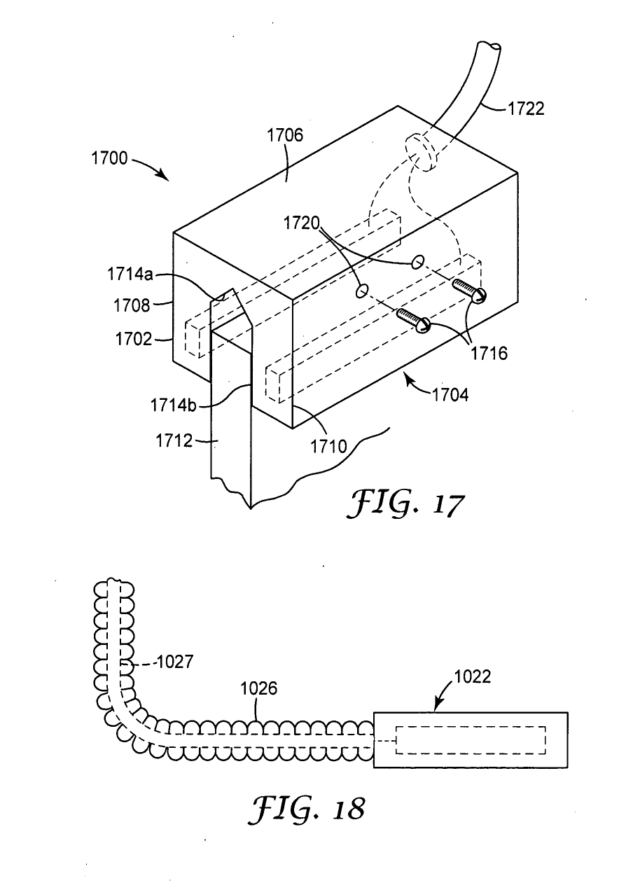

FIG. 10 illustrates a low-cost retrofitting approach for adding low voltage probes to a fryer and food container, such as a fryer basket. In this embodiment, the probe assembly 1020 may be connected to the food basket so as to be movable therewith. The probe assembly 1020 is similar to the low voltage-probe described above. The probe assembly 1020 includes a probe unit 1022 that may be connected to the fryer basket 1024 by any suitable connection 1025. The probe unit 1022 is held by a probe holder 1026 and the probe holder is attached to a flexible hose 1028. The probe holder may be a bendable tube that may be bent to a desired shape for holding portions of the probe unit assembly therein and at any orientation. FIG. 18 illustrates an exemplary embodiment of a probe holder 1026 for holding a probe 1022 in a desired orientation. The probe holder 1026 may be a bendable and shape retentive corrugated tube 1027 that may be made of a suitable material, such as stainless steel, aluminum and the like to satisfy the environment that it is placed. Accordingly, the probe may be suitably retained in and spaced in the cooking oil at desired orientations. Such a shape retentive corrugated tube is commercially available.

A variety of suitable materials may be provided for mounting the probe units in the vats, such as flexible holders or combinations of flexible and bendable materials. The probe holder 1026 may held as by any suitable means including, but are not limited to brackets, adhesive tapes, or the like to the fryer basket 1024. The probe unit 1022 when supported may be spaced from the fryer basket and thus the food. The probe unit 1022 may be placed in any orientation and spacing from the frying basket 1024. By being spaced from the basket 1024, the probe unit 1022 does not physically interfere or block flow of the cooking medium therethrough. In some cases, the probe may be attached to the fryer basket. The probe holder 1026 may be attached to a flexible hose 1028 and allows the probe unit 1022 to be inserted and removed from the fryer vat as the fryer basket is placed in and removed therefrom. This allows economical retrofitting of existing fryers that fit closely to the walls defining the vat. The probe assembly 1020 may include an external source of power 1030 coupled to the probe unit 1022. Alternatively or additionally, the probe assembly 1020 may include an internal source of power, such as a battery (not shown), thermocouple (not shown) or the like within the probe unit 1022.

FIG. 11 illustrates an embodiment in which the probe assembly 1120 including probe unit 1122 is mounted by a fixture 1128 to a wall 1142 instead of fryer baskets 1124. The fixture 1128 includes a bracket 1144 that is mounted to be secured to an edge portion of the vat wall. The probe unit 1122 is positioned so as to be clear of interfering with insertion and removal actions of the fryer basket. The probe unit 1122 is adapted to be spaced from the fryer basket when the latter is within the fryer chambers. The probe unit 1122 is connected by a flexible hose to an appropriate source of power. While the bracket 1144 is shown capable of suspending the probe assembly, any suitable bracket or fastening device is contemplated so as to fixedly or removably hold the probe assembly in the desired location. It will be appreciated that the probe unit 1122 may be held in contact with the vat walls.

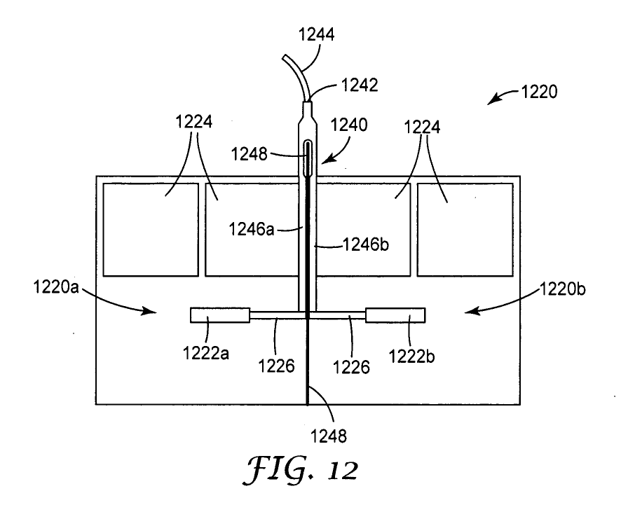

FIG. 12 illustrates another embodiment of a probe assembly 1220 for supporting probes and which includes a generally T-shaped fixture 1240 is mounted to a wall 1248 separating two vats 1220a and 1220b. The fixture 1240 is adapted for holding a pair of probe assemblies 1222a and 1222b in respective ones of the vats away from the fryer baskets 1224 when the latter are used in the fryer. The probe assemblies are similar to those described above. The T-shaped fixture 1240 includes a pair tubular channel 1246a and 1246b, one for receiving and protectively covering each of flexible cables (not shown) associated with the probe assemblies in each vat. In this regard, the tubular channels are joined to the tubular probe holders 1226, similar to the probe holders noted above, so that the probe assembly may extend through both. Accordingly, each probe assembly 1222a and 1222b may be mounted in each of the vats. Each of the probe assemblies in the tubular channels may exit an opening 1242 be connected to a cable 1244 that supplies power separately to each of the probe assemblies under the control of respective ion generator controllers that may in turn be under the control of a microcontroller, such as noted above. Accordingly, different probes may be operated independently for independently controlling the oxidation and hydrolysis of cooking media in different vats. For example, different rates of electron discharge for inhibiting the oxidation and hydrolysis for different food treating media may be obtained by varying the voltage to the different probe assemblies.

Table 1 below depicts the results of frying French fries in a commercial frying oil according to the present description by comparing free fatty acid content of frying oil in two adjacent vats. The vats and probes used are comparable to vats 912 and 914 illustrated in FIG. 9.

TABLE 1

| | Days | 0 | 1 | 2 | 3 | 4 | 5 | 6 | 7 | 8 |
|---|---|---|---|---|---|---|---|---|---|---|
| Vat | No | 0.4 | 2.63 | 6.81 | 5.39 | 6.00 | 5.84 | 6.93 | 9.40 | |
| 1 | Probe | | | | | | | | | |
| | With 1 | 0.4 | 0.63 | 1.52 | 2.66 | 3.65 | 5.63 | 6.72 | | |
| | Probe | | | | | | | | | |
| | Vat 1 | 0.4 | 1.50 | 2.28 | 3.52 | 4.81 | 5.29 | 6.03 | 7.20 | 6.80 |
| | With 2 | | | | | | | | | |
| | Probes | | | | | | | | | |
| | Vat 1 | | | | | | | | | |
| Vat | No | 0.4 | 3.70 | 4.95 | 6.95 | 4.62 | 7.20 | 6.27 | 8.59 | |
| 2 | Probe | | | | | | | | | |
| | With 1 | 0.4 | 2.00 | 3.02 | 4.18 | 3.66 | 5.63 | 6.72 | | |
| | Probe | | | | | | | | | |
| | No | 0.4 | 1.0 | 1.45 | 2.67 | 3.35 | 6.01 | 6.71 | 7.10 | 6.90 |
| | Probe | | | | | | | | | |
| | but in | | | | | | | | | |
| | contact | | | | | | | | | |
| | w/vat | | | | | | | | | |
| | having 2 | | | | | | | | | |
| | Probes | | | | | | | | | |

Table 1 illustrates the advantages derived by the present description. In making reference to Table 1 reference is also made to FIG. 9. Vat 1 may be considered vat or chamber 912 and Vat 2 may be considered vat or chamber 914. The Table was prepared by deep frying fries in both vats. Under one test condition, no probes were used in Vats 1 and 2, while frying the French fries. Under another test condition, one low voltage probe was suspended in each separate Vat 1 and 2. In a third test condition, two probes were placed in Vat 1, but in contact with the walls defining the vat, which walls were in electrically conductive relationship with the walls of Vat 2. However, no probe was used in Vat 2. The values in the charts were taken at the end of testing day by using testing as noted above. All the probes used were ion generator units that were commercially available from Rejuvenoil Hoei America, Inc.

For conducting the comparison, substantially the same testing conditions were used in both vats except were noted. The free fatty acid content was measured using a known testing process as described above. A cooking oil, such as noted above, was used and is commercially available. During deep frying operations, the cooking oil was heated to a temperature in a range of about 325° F. to about 375° F. At the end of each cooking day, the cooking oil was drained from a vat and filtered through a known filtration system associated with the fryer. The deep fryer was a commercially available deep fryer. The next day fresh cooking oil replenished that lost the previous day.

FIG. 13 A illustrates a plot of the test results taken from Table 1, noted above, comparing the free fatty acid content on a percentage of weight basis relative to cooking or frying oil. For purposes of illustration only a free fatty acid value of 7% may be a level considered unacceptable. Plot line 1310 illustrates that with no probe in Vat 1 after two days of frying a free fatty acid value of 6.81% was reached. As is known practice, fresh frying oil was also added beyond replenishment after the second day. On the third day the free fatty acid value was 5.39%. On the fourth day the free fatty acid value was 6.00%. Additional fresh cooking oil was also added beyond replenishment after the fourth day. On the fifth day the free fatty acid value was 5.84%. On the sixth day the free fatty acid value was 9.40%. Then the entire batch of oil was discarded. Plot line 1320 illustrates the free fatty acid values using the one aspect of the present description using one probe suspended in Vat 1 and another is suspended in Vat 2. Plot line 1330 depicts the free fatty acid value when Vat 1 has two ion generators suspended in it while no ion generator is immersed in Vat 2. In this situation both ion generator probes 950a, 950b were in electrical contact with the vat walls.

It is observable that the free fatty acid values in Vat 1 with a single probe are significantly lower that the free fatty acid values without the probe. Beyond normal replenishment, essentially the same frying oil batch was used in the frying of the French fries throughout six days. Surprisingly, with two probes in Vat 1 and no probe in Vat 2, the free fatty acid values were generally similar to the test condition wherein one probe is in Vat 1 and one probe is in Vat 2. However, with two (2) probes in Vat 1, the free fatty acid value for day 7 was about 7.2%. In this regard, two generating probes 950a, b in Vat 1 are electrically connected to the conductive walls defining Vats 1 and 2. Accordingly, it has been determined that the probe need not be placed in both chambers to effect an oxidation and hydrolysis. In this situation, the present description makes use of the capacitive effect brought about by the noted ion generator and distributes the electrons to the cooking oil along the entire surface of the conductive material that is common to both of the vats.

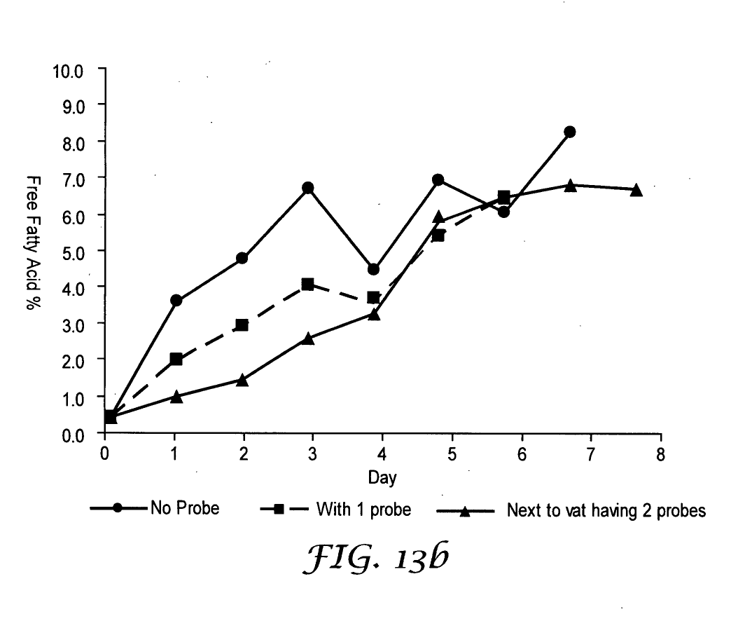

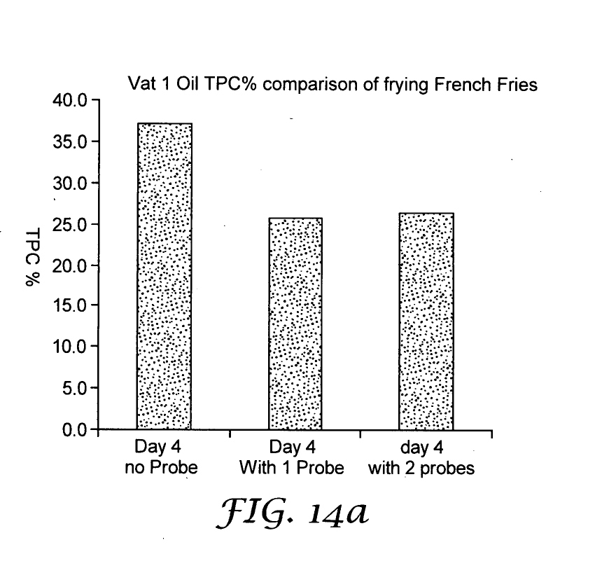

FIGS. 14 A and B depict bar charts respectively illustrating the advantageous results achievable by of the present description. FIG. 14A illustrates that for vat 912 the values of TPC % percent by weight over a period of four days were as follows: for no probe the TPC value was exceedingly higher than for the same vat having one probe or for two probes. Both the TPC % values for 1 probe and for 2 probes were approximately the same. FIG. 14B illustrates the TPC readings for vat 914. The bar for no probe bar showed relatively high TPC % values compared to the bar for one probe in the vat 914. It will be recalled that in the latter situation, one probe was used in both vats 912 and 914. The other bar showed that the TPC % values after four day, with vat 912 having two probes while vat 914 does not have any probe, were comparable to the values when one probe was present in each of the vats 912 and 914. It is believed that because of the capacitive distribution of electrons from the ion generator probes 950a,b and the electrical conduction of electrons by the conductive walls of the vats both vats 912 and 914 had comparable reductions in TPC content. It will be appreciated that with one probe in each vat and with two probes in one vat only there were significant reductions in the TPC content relative to no probes being used. As a result, the life of the cooking medium was extended. Accordingly, significant savings may be made in terms of the consumption of cooking oil.

Alternatively or additionally, a wide variety of other exemplary embodiments of a stabilizing unit or assembly are contemplated by the present description. In general, these other approaches, as with the former constructions, enhance the overall versatility of positioning probes relative to a vat and/or a cooking oil medium in order to effectively and efficiently inhibit degradation of the oil medium.

Reference is made to FIG. 15 for illustrating another exemplary embodiment of a stabilizing unit or assembly 1500 of the present description. The stabilizing unit 1500 may include at least one probe 1502 that is similar to those described above. The probe 1502 may include a housing assembly 1504 that surrounds semiconductor material 1505 housed therein. The semiconductor material 1505 may include the material(s) described above for generating low voltage. The housing assembly 1504 may have a generally parallelepiped construction with two opposite major and generally rectangular and planar plates 1506 a, b. The major surface 1507 of the plate 1506a is adapted to intimately engage a coupling assembly 1512 a vat wall 1508 and is sized and shaped for transferring a sufficient amount of electrons for the purposes intended. While a parallelepiped construction is depicted for the housing assembly, it will be appreciated that other configurations and sizes may be used. For example, a thin and generally planar sheet or plate like member (not shown) may be provided in intimate engagement with a vat wall. The housing assembly 1504 may be made of a suitable conductive material(s). The suitable material that may be used includes, but is not limited to, stainless steel and the like. The present description describes that the low voltage probe may be totally or partially submerged in a cooking oil 1510 or not submerged at all. Accordingly, the present embodiment also contemplates that the housing assembly 1504 may be sized so that bottom of the plates 1506 a, b (as viewed in the drawing) are spaced above the cooking oil 1510 during usage, or partially submerged (not shown) or totally submerged (not shown) in the cooking oil. Alternatively, because of the clip the housing assembly may be positioned on an exterior surface of the vat.

To insure an intimate engagement for the purposes intended, the illustrated embodiment makes provision for the stabilizing unit 1500 including a coupling assembly 1512 that is shaped and constructed to releasably secure the housing assembly 1504 in intimate engagement with the vat wall 1508. As a result, electrons from the probe unit 1502 pass to the conductive portion of the vat wall 1508 in an amount sufficient to effectuate the contemplated stabilizing of the cooking oil. In the illustrated exemplary embodiment, the coupling assembly 1512 may be a generally U-shaped spring-biased clip member 1512 made of a resiliently flexible material that is also compatible to the cooking oil, such as stainless steel, aluminum, or other suitable materials. The clip member 1512 has a proximal end portion 1514 attached to the major surfaces 1506a and the opposing distal end 1516 may be flexed to accommodate the thickness of the vat wall 1508. In addition, the clip member 1512 may be made of an electrically conductive material for enabling the clip to also transfer electrons from the major surface 1506a to a vat wall. The foregoing arrangement is versatile in terms of a user be able to retrofit a variety of vats at a variety of locations with a probe unit. While a single clip member is illustrated, it will be understood that the present description envisions other equivalent constructions for a clip, such as including but not limited to clasps, clamps, and the like as well as the use of more than one clip. While this embodiment is disclosed as releasable type, it will be understood that a suitable fastener(s) may be used to secure the clip to a vat wall in a more permanent fashion. Also, a tube 1522 containing a wire (not shown) for energizing the material may be welded to the housing assembly to prevent external vapors from condensing on the tube and entering the interior of the housing assembly and adversely affecting components of the housing assembly and thereby cause malfunctioning. Although not shown the present description envisions the use of a thin plastic absorbent material interposed between the surface of the proximal portion 1514 and the vat wall to absorb any cooking oil vapors and hence diminish build-up of such materials therebetween. In situations wherein the tube 1522 may not be welded, it may be connected to a lateral side of the housing assembly 1504 instead of located at the top of the housing assembly. As such, there is less of a likelihood of condensation of cooking oil vapors dripping along the length of the tube and, under the influence of gravity, possibly enter the housing at a joint (not shown) between the housing assembly and the tube. Accordingly, shelf-life of the stabilizing unit may be increased since moisture causing malfunctions are to be minimized.

FIG. 16 illustrates another exemplary embodiment that is similar to FIG. 15. In the present exemplary embodiment, a different configuration is provided for a stabilizing unit or assembly 1600 that includes a probe 1602 similar to the previous embodiment and a coupling assembly 1604 similar to the previous embodiment. The probe 1602 includes a housing assembly 1606 having a parallelepiped construction that may be smaller in cross-sectional area than the previous embodiment and includes an material 1607 of the kind noted above that is energized as described above for generating a sufficient amount of electrons to be transferred as described. A wall 1608 thereof may be attached to the coupling assembly 1604, in any suitable manner, so as to be in intimate engagement therewith, whereby electrons from the material may be transferred. In particular, the coupling assembly 1604 may be a generally U-shaped member having a pair of opposing spring-like leg portions 1610a, b having distal ends configured as illustrated. One leg portion 1610a may have the housing assembly 1606 appropriately attached adjacent to a distal end thereof. The materials of the housing assembly and the coupling assembly may be electrically conductive. The length of the leg portion 1610a generally determines whether the housing assembly is above, partially immersed, or completely submerged in the cooling oil. Alternatively, the probe 1602 may be arranged so that the leg portion is not in the vat, but on the outside or exterior wall of the vat. In situations wherein the tube 1622 may not be welded, it may be connected to a lateral side of the housing assembly 1606 instead of located at the top of the housing assembly. As such, there is less of a likelihood of condensation of cooking oil vapors dripping along the length of the tube and, under the influence of gravity, possibly enter the housing at a joint (not shown) between the housing assembly and the tube. Accordingly, shelf-life of the stabilizing unit may be increased.

While a generally U-shaped clip is illustrated for the coupling assembly, other equivalent constructions may be utilized to arrive at suitable releasable connections. Alternatively, the coupling assembly may include different kinds of attaching and fastening elements for fixedly securing the coupling assembly to a vat wall.

FIG. 17 illustrates a stabilizing unit or assembly 1700 made according to another exemplary embodiment of the present description. The stabilizing unit 1700 may include, in an integral manner, a pair of enclosed a pair of depending low voltage probe portions 1702, 1704 that may be included in an integral or one-piece housing assembly 1706. As with the other embodiment, the housing assembly 1706 is made of a suitable conductive material for transferring the electrons. A semiconductor material of the kind described above may comprise the material for the low voltage probe portions. The housing assembly 1706 may have generally U-shaped construction with two generally parallel and spaced apart opposing leg portions 1708, 1710. The leg portions 1708 and 1710 are adapted to house the probe portions 1702 and 1704; respectively. The leg portions 1708 and 1710 are adapted to straddle and engage opposing sides of a vat wall portion 1712, such as illustrated. In this regard, threaded fastening elements 1716 or the like may be provided that may threadedly cooperate with openings 1720 formed in the leg portions and openings (not shown) in openings (not shown) in the wall for firmly securing the leg portions. Their respective surfaces 1714 a, b may have a wide variety of shapes and sizes for effectuating the transfer of electrons in a sufficient manner consistent with the teaching of the present description. The leg portions 1702 and 1704 may extend downwardly by an amount that varies. For example, one or more of the leg portions 1702 and 1704 may be of such a length that the electron emitting probe portions are totally submerged within the cooking oil. Other embodiments envision that one or more of such portions may be partially submerged or completely not submerged within the cooking oil. In situations wherein the tube 1722 may not be welded, it may be connected to a lateral side of the housing assembly 1706 instead of located at the top of the housing assembly. As such, there is less of a likelihood of condensation of cooking oil vapors dripping along the length of the tube and, under the influence of gravity, possibly enter the housing at a joint (not shown) between the housing assembly and the tube. Accordingly, shelf-life of the stabilizing unit may be increased.

Reference is made to FIG. 19 for illustrating another exemplary embodiment of the present description. In this embodiment, provision is made for a system 1900 that controls a plurality probe assemblies 1902a-n (collectively, 1902), each of which is adapted to be associated with a fryer system 1902 containing a plurality of fryer vats 1904 a-n (collectively, 1904) defining respective chambers 1906 a-n (collectively, 1906) into different cooking oil media (not shown) may be added so as to, for example, treat different food items (not shown). The vat walls as with those described earlier may be made of an electrically conductive materials consistent with the spirit and scope of the present description. The probe assemblies may be similar to those described above for introducing electrons into the cooking oils. The probe assemblies 1902 c, n, for example, may be sized to engage side walls 1907 of respective vat walls; in a similar fashion as probe 950f (described in FIG. 9). The probe assemblies 1902 a, b may be arranged to be directly suspended to be above the cooking oil (not shown) or may be partially or totally submerged. All the probe assemblies 1902 may be suspended at various heights to achieve a desired location relative to the cooking oil. While a single probe assembly is associated with each vat, it will be understood that other suitable probe assemblies may be utilized for each vat or no probe assemblies need be applied. Also, while not shown, the probe assemblies may be connected to an exterior wall surface of the vats. The probe assemblies may have different configurations and sizes as well. All of the probe assemblies 1902 are at a distal end of a tube 1908 that carries the wire to the material(s) (not shown) in the probe assemblies. At a proximal end of the probe assembly a coupling 1910, such as an internally threaded coupling, may be used to cooperate with an external fitting (not shown) connected to a central conduit 1912 that serves as part of a supporting assembly for supporting the probe assemblies as described herein. While a threaded connection is used to join the central conduit 1912 and the coupling 1910 other suitable mechanisms and approaches may be used. Alternatively, the probe assemblies 1902 may be fixedly connected to the central conduit 1912. The central conduit 1912 may extend from one end of the vat to the other and is secured by clamps 1914 a, b or other suitable and similar retention system. The central conduit 1912 may be a rigid material, such as a suitable metal, plastic, and the like. The central conduit 1912 may have externally threaded fittings (not shown) that cooperate with internal threads of the coupling 1910. Such fittings allow passage of the wires for each probe that are carried internally of the central conduit 1912. While the central conduit is illustrated as a single elongated member, the central conduit may be made of segments and/or be made of flexible materials and/or have non-linear portions. A controller 1920 may be coupled through the wiring 1918 provided to control each of the probe assemblies so as to vary the voltage as noted as well as to turn ‘on’ and “off” power to the probe assemblies. The controller may have suitable manually and/or automatically controlled devices that allow each of the probe assemblies to be controlled consistent with the spirit and scope of the present description.

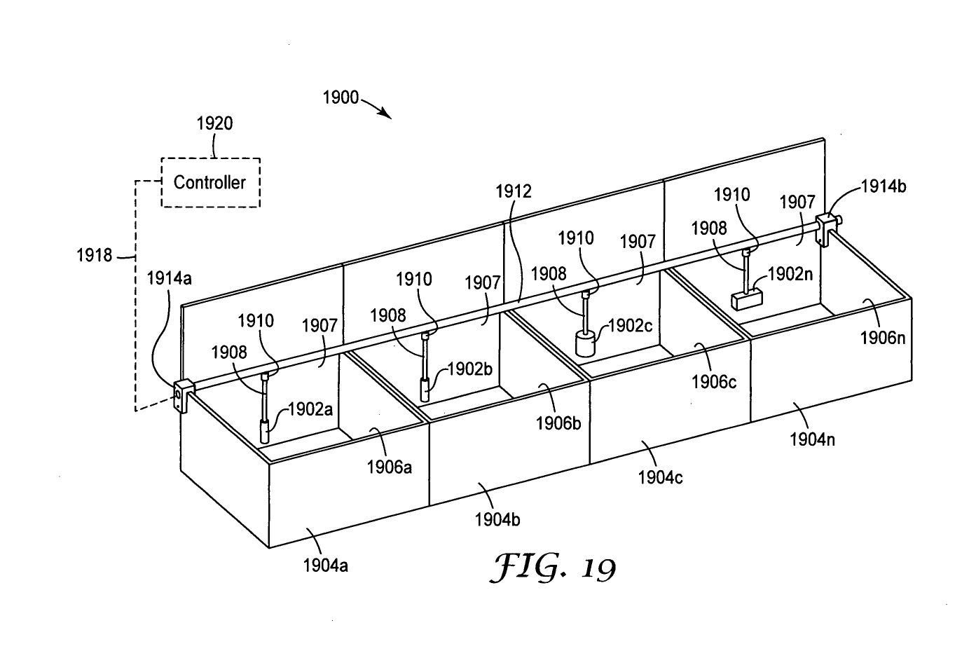

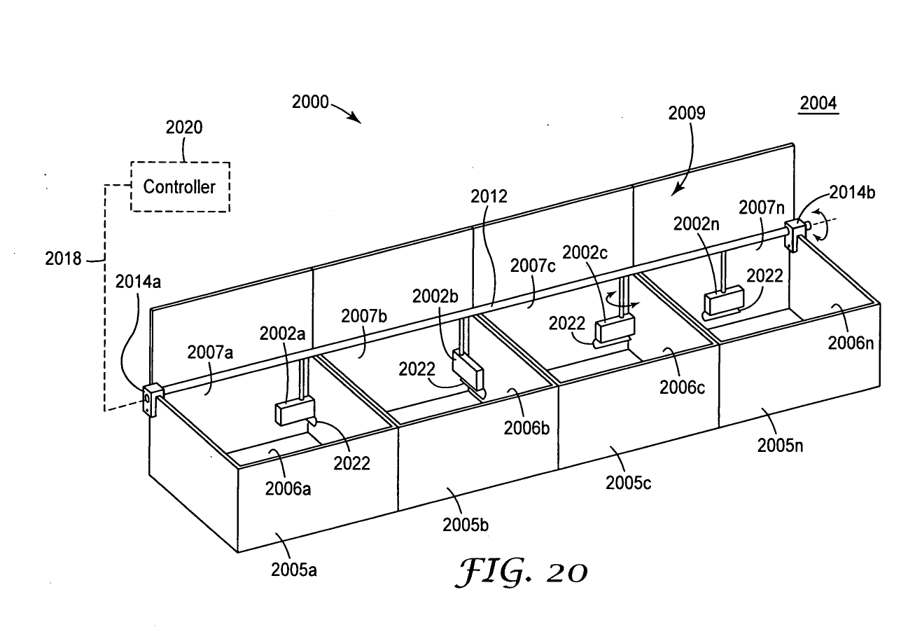

Reference is made to FIGS. 20-28 for illustrating exemplary embodiments used for installing and controlling a system 2000 including probe assemblies 2002a-n (collectively, 2002) in a manner that allows for retrofitting in accordance with the present description.

FIG. 20 illustrates the system 2000 including the probe assemblies 2002, as installed, that may be used in combination with a fryer system 2004 including a plurality of frying vats 2005a-n (collectively, 2005). As noted, the present description is usable in combination with many types of food treating systems, such as cooking/frying systems having different sizes and shapes as well as different operating components (not shown), such as automatic fryer baskets that operate therewith. As will be apparent, the system 2000 and method of installing the same enable versatile, low cost, and yet easy retrofitting of a fryer system or the like which utilizes multiple probe assemblies 2002a-n (collectively, 2002). The probe assemblies 2002 are adapted to be positioned in predetermined locations in multiple preexisting frying vats 2005, such that the probe assemblies meet operating conditions that are consistent with the principles of the present description. In addition, the system 2000 facilitates easy retrofitting installation as well as facilitates the cleaning of the vats as well as the probe assemblies themselves. The probe assemblies 2002 may be similar to those described earlier for introducing low voltage to the vat and cooking oil.

The probe assemblies 2002 may be similar to the probe assemblies noted above in regard to the previous exemplary embodiments. One difference is that the probe assemblies 2002 may have a probe body 2010 having a generally paddle type parallelepiped configuration, as illustrated and be constructed to be rotated about a vertical axis through a limited amount, such as by about 90° so that a probe assembly may be moved into selective engagement with, for example, orthogonally disposed sidewalls of a vat. Such rotation of the paddle-like probe body 2010 adds to the versatility of the present embodiment being retrofitted to a wider variety of vats.

The frying vats 2005 may define respective chambers 2006a-n (collectively, 2006) into which cooking oil media (not shown) may be added to treat, for example, different food items (not shown). Internal vat sidewalls 2007a-n (collectively, 2007) of each vat are like those described earlier and generally have an electrically conductive surface over at least a major portion thereof. The probe assemblies 2002 may be adapted to engage the sidewalls 2007 in a similar fashion as probe 950f (described in FIG. 9). Also, the probe assemblies 2002 may be arranged to be partially or totally submerged (not shown) in the cooking oil while being in electrical contact with the sidewalls. While a single probe assembly is illustrated with each vat, it will be appreciated that more than one probe assembly can be suspended or otherwise connected for a single vat. Also while not shown, the probe assemblies 2002 may be attached to the outside wall of each vat. The probe assemblies 2002, as noted, may have different configurations, such as but not limited to the configurations of the kinds previously illustrated. It will be appreciated that the probe assemblies 2002 of the present description are not limited to the sizes and configurations of the probe assemblies illustrated.

All of the probe assemblies 2002 may be coupled as by being threadedly connected to a distal end of a vertical supporting tube 2008 that carries the wires to the ion generating material(s) (not shown) in a probe assembly 2002. As illustrated in FIG. 22, a proximal end of the supporting tube 2008 is provided with a threaded connection 2008a for threaded engagement to a threaded opening (not shown) in a generally horizontally disposable central supporting conduit member 2012. The supporting tube 2008 and the central horizontally disposable supporting conduit member 2012 serve as part of a supporting assembly 2009 that supports and the probe assemblies 2002, which may be suspended therefrom. Such a supporting assembly 2009 includes the tubes 2008 generally extending from the conduit member 2012 and acting as extending supporting members arranged for depending in a generally upstanding relationship to and along the length of the horizontally disposable supporting member in response to the horizontally disposable supporting member being supported on a vat

Each of the supporting tubes 2008 may be threadedly connected at their distal ends 2008b to an internal threaded fitting (not shown) in a top portion of a corresponding one of the probe assemblies 2002 adjacent an end thereof. As such, a user or operator may rotate each probe assembly by a limited specified amount relative to a vertical axis of the tube, whereby, for example, a probe assembly 2002 placed in a corner of a vat may be selectively moved into engagement with orthogonally disposed sidewalls 2007 of a vat. Alternatively, the distal end portion 2008b may be coupled by other than threaded couplings to the probe assemblies 2002 for allowing relative rotation thereof. In addition, the present description envisions that the distal ends 2008b may be welded to the probe assemblies. The present description envisions one exemplary embodiment in which a paddle shaped probe assembly 2002 may be rotated so that in one orientation, a probe assembly sidewall may be engaged with one of the respective vat sidewalls, and when rotated by, for example, 90 degrees another opposing probe assembly sidewall may be engaged with another one of the vat sidewalls. The present description envisions other angular ranges for selectively rotating a probe assembly so that opposing wall portions of a single probe assembly may engage corresponding different vat sidewalls. The probe assemblies 2002 themselves may not have planar sidewalls, but walls that bow outwardly.

The system 2000 includes the central supporting member 2012 or central supporting conduit member 2012 that may be constructed as a tubular member that, as noted in an earlier embodiment, carries probe wires 2018 to and from each of the probe assemblies 2002 to a controller 2020. The controller 2020 may be similar to controller 1920 (FIG. 19) in terms of its construction and functionality and as such a description thereof is not believed necessary for this embodiment. The central supporting member 2012 or conduit 2012 may be constructed with threaded openings (not shown) at spaced apart linear intervals for threaded cooperation with the tubes 2008 connected to probe assemblies 2002. Alternatively, the central supporting member 2012 may be made from several different attached together components in end-to-end relationship. The central supporting member 2012 may be supported or coupled by brackets 2014a, b or the like that are connectable to ends of the central supporting member 2012 and the ends of the fryer system 2004 as illustrated. The brackets 2014a, b may be of the type that enables the central supporting member 2012 to be rotated about its longitudinal axis. As such, rotation enables the probe assemblies 2002 attached thereto to be rotated up and away from the vat sidewalls 2007. In this fashion, the frying vats 2005 may be more easily cleaned as well as the probe assemblies themselves. A wide variety of brackets and the like may be used for supporting the central supporting member 2012 relative to a vat system. Alternatively, the central supporting member 2012 may be merely supported on the vat without the aid of a bracket assembly.

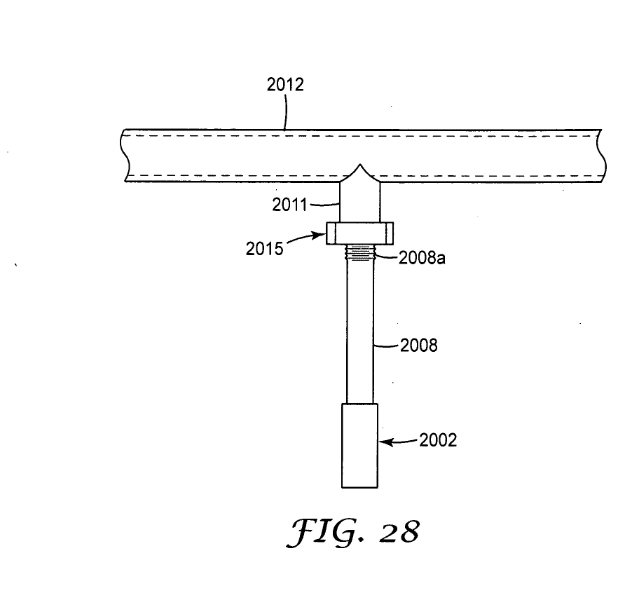

Reference is made back to the threaded couplings 2008a, b of the supporting tube 2008. For example, either one or both of the threaded couplings may permit the probe assemblies to rotate by a limited amount, such as by a ¼ of a turn (90 degrees), even after otherwise firmly securing the probe assemblies in a desired orientation. For example, one suitable type of threaded coupling for achieving this is known as a tapered threaded coupling, such as a National Pipe Thread Tapered (i.e., NPT) pipe threaded fitting and is readily commercially available. The taper of the threads allow formation of a seal when torqued. This kind of threaded coupling is not only economical, but also provides for a sealing effect in sealing against cooking oil, vapors and the like entering the central supporting member 2012. While such threaded pipe fittings or couplings are utilized, other kinds of threaded fittings or couplings may be used to permit selective rotation of the probe assemblies 2002 while facilitating securing and sealing the latter. These other types of threaded couplings may include, but are not limited to UNC and UNF Series threaded couplings. Besides tapered threaded couplings providing sealing, sealing may be effectuated by gaskets (not shown) and O-rings (not shown) or other similar device. Alternatively, instead of the threaded couplings 2008a being engageable with threaded openings in the central supporting member 2012, provision is made to another coupling arrangement. In this regard, reference is made FIG. 28 for illustrating a coupling including a hub 2011 that may be welded to supporting member 2012. The hub 2011 includes a central internally threaded opening (not shown) for allowing the probe wires to pass therethrough. The tube 2008 has its threaded end 2008a in threaded engagement with the central internally threaded opening hub. A lock nut 2015 could be attached for rotation on the threaded end 2008a. By loosening the lock nut 2015, the tube and paddles may be rotated to any desired position. In a known manner, the lock nut 2015 would be tightened and upon engaging the hub 2011, the tube 2008 is prevented against further rotation. As such, the tube 2008 may be locked in any desired angular orientation relative to its longitudinal axis, thereby locking the probe assembly 2002 in desired orientation engaging a vat sidewall. Also, the present description envisions other approaches including snap fittings and the like for providing the coupling.

The probe assemblies 2002 may include one or more spring-biased electrical conductor elements 2022, such as an electrically conductive spring clip 2022. The electrically conductive spring clips 2022 are constructed of a suitable electrically conductive material that also satisfies requirements of being able to be used with food. One such material is stainless steel. Of course, other suitable materials may be provided consistent with the teachings of the present description. The electrically conductive spring clip 2022 may have a bowed configuration, such as the type illustrated in as illustrated in FIGS. 23, 24, 26 and 27 for extending away from a vertical plane of a probe body of a probe assembly. In FIG. 27, the spring clip 2022 may be connected to an end portion of the probe instead of along a longitudinal sidewall thereof. The electrically conductive spring clip 2022 may assume other orientations so long as the configurations and sizes are selected to enable the probe assembly to engage and be electrically conductive with the vat sidewalls. The bowing of the spring clip compensates for clearances or misalignments between the probe assemblies, in their intended orientations, and the vat sidewalls when the probe assemblies have been oriented as desired to contact a vat sidewall. Also, while a single electrically conductive spring clip is illustrated as being attached to a sidewall of a probe housing, more than one electrically conductive spring clip may be provided. While in the illustrated exemplary embodiment the spring clips are disposed below a probe housing, the spring clips may be arranged in other positions relative to the probe assemblies, such as being disposed immediately adjacent the probe housing. While the illustrated exemplary embodiment of the electrically conductive member may be a spring clip, such electrically conductive member need not be spring biased. Accordingly, the spring-biased electrical conductor element 2022 compensate for any misalignments of the probe assemblies not being properly aligned to make contact with the wall portions for ensuring transferring the low voltages of the probe assembly thereto for the advantages noted above.

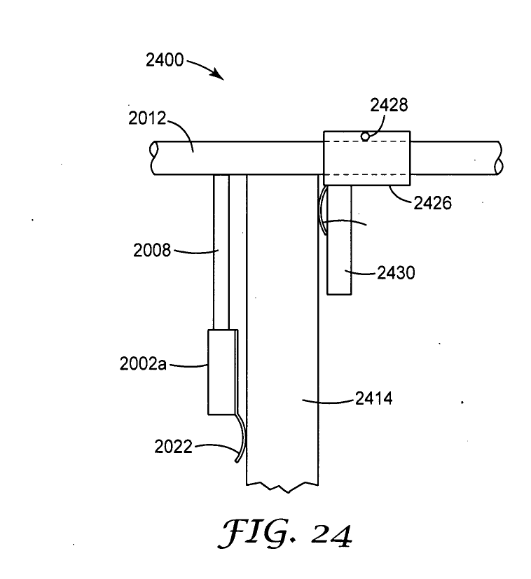

Reference is made to FIG. 24 for illustrating that the system for installing may include a retaining assembly 2400 that is mounted on the central supporting member 2012 so as to be placed in close proximity of a vat partition wall 2414. The retaining assembly 2400 serves to generally retain the central supporting member 2012 in a stationary position so that a probe assembly 2002 in engagement with an adjacent vat sidewall will be retained in such position. It will be appreciated that the retaining assembly 2400 may have several different constructions and in the exemplary illustrated embodiment includes a sleeve 2426 slidable on the central supporting member 2012. The sleeve 2426 is positioned by a set screw 2428. Depending from the sleeve 2426 is a retaining element 2430 that may include a spring biased clip 2432 adapted for engagement with the vat partition wall 2414 while the probe assembly is engaged with the vat sidewall. As such, an arrangement is provided that enables stabilizing a probe assembly in a desired position.

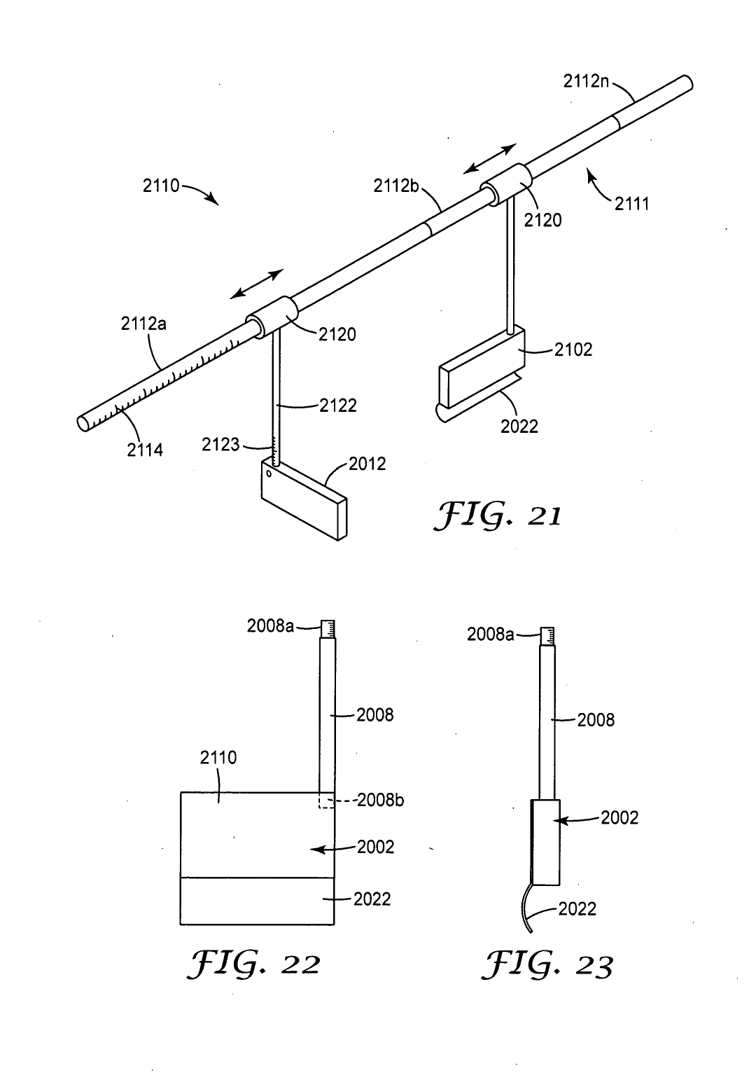

Reference is now made to FIG. 21 for illustrating a template assembly 2110 that may be used consistent with the present description to preliminarily determine an appropriate construction for the system 2000 (FIG. 20) that may be retrofitted to a particular sized fryer system. The actual retrofitted installation system 2000 may be based on use of the final construction of the template assembly 2110. The template assembly 2110 of the present description may comprise a template supporting member 2111 comprised of a plurality of interfitting tubular pipe supporting portions 2112a-n or supporting segments 2112a-n, (collectively, 2112) that may be of different lengths. In this regard, opposing longitudinal end portions of each of the different supporting template segments 2112 may be tapered (not shown) for appropriate sliding frictional engagement within mating tubular supporting template segments 2112. Different length supporting template segments 2112 may be used to be selectively connected in end-to-end relationships to arrive at an overall length that matches the configuration of the vat that is to be retrofitted with the system 2000. Ideally, the supporting template segments 2112 may have different lengths to enable the template assembly 2110 to be more easily adjusted to a variety of vats regardless of the size and spacing of the vats to be retrofitted. While this embodiment discloses the segments are in coupled relationship by physical contact with each other, it will be appreciated that additional coupling devices, for example, brackets and the like may be used to join separate template segments whether or not the latter rely on physical contact with each other.

The template segments 2112 may have measuring gradations markings 2114 provided for measuring purposes. Besides gradation markings other approaches may be used to measure locations along the template supporting member 2111 at which a probe assembly 2102 is be positioned to engage a vat sidewall. Alternatively, the template supporting member 2111 of the template assembly 2110 may not be made of several components, but include a single supporting member 2111 such as a bar having a variety of cross-sectional shapes.

For the template assembly 2110 to determine the proper position of the probe assemblies 2002 relative to the vats, the template assembly 2110 may include one or more supporting devices 2120, as a sleeve 2120, is slidably movable along the length of the supporting member 2111. The supporting device may have a single probe assembly 2102 coupled thereto and depending therefrom by a supporting element 2122 or support rod 2122. It will be understood that a variety of other constructions may be provided, instead of a slidable sleeve, to provide replaceable mountings of the probe assembly 2102 on the supporting member 2111, so that the probe assembly may be adjusted to engage a vat sidewall. In use, the sleeve 2120 may be slid along the supporting member 2111 until the probe assembly 2102 abuts a sidewall of a vat (not shown) to be retrofitted. This abutting position will indicate at what point along the length of the supporting member 2111a probe assembly 2002 should be placed on the central supporting member 2012 so that it may engage a vat wall when retrofitted. It will be understood that the supporting member 2111 is generally adapted to be similar in length to the central supporting member 2012 of the system 2000 for installing. A user may take measurement and duly record such locations and measurements for future use in terms of being able to accurately locate a probe assembly 2002 on the central supporting member 2012. Such a process is repeated until the locations of all the probe assemblies 2102 in their vat abutting positions are recorded. It will be further appreciated that the probe assembly 2102 need not be an actual probe assembly, but an object or replica designed to resemble and serve as a substitute for an actual probe assembly. Such a substitute replica or probe assembly may include a spring clip electrical connector as well. Also, the probe assembly 2102 may be vertically movable along the support rod 2122. The support rod 2122 may have markings 2123 thereon that allow the replica probe assembly 2102 to be vertically adjusted relative thereto for determining the distance a probe assembly is to be suspended into a vat.

Accordingly, the template assembly 2110 allows the ready and easy construction of a mock-up installation assembly that resembles and is sized similarly to form an installation system usable for retrofitting purposes. As such, an operator may take and measurements for an actual installation system in a relatively straightforward manner by merely duplicating the template assembly. It will be appreciated that a retrofitting procedure using the template assembly 2010, as noted, advantageously may be made without installers expending a significant amount of time taking measurements coupled with a relatively high degree of accuracy of probe placement. Further the template assembly may be locked or fixed with its final orientation after the one or more depending elements have been added to the elongated supporting device at locations corresponding to desired locations of probe assemblies, whereby the template assembly may be shipped for manufacturing.

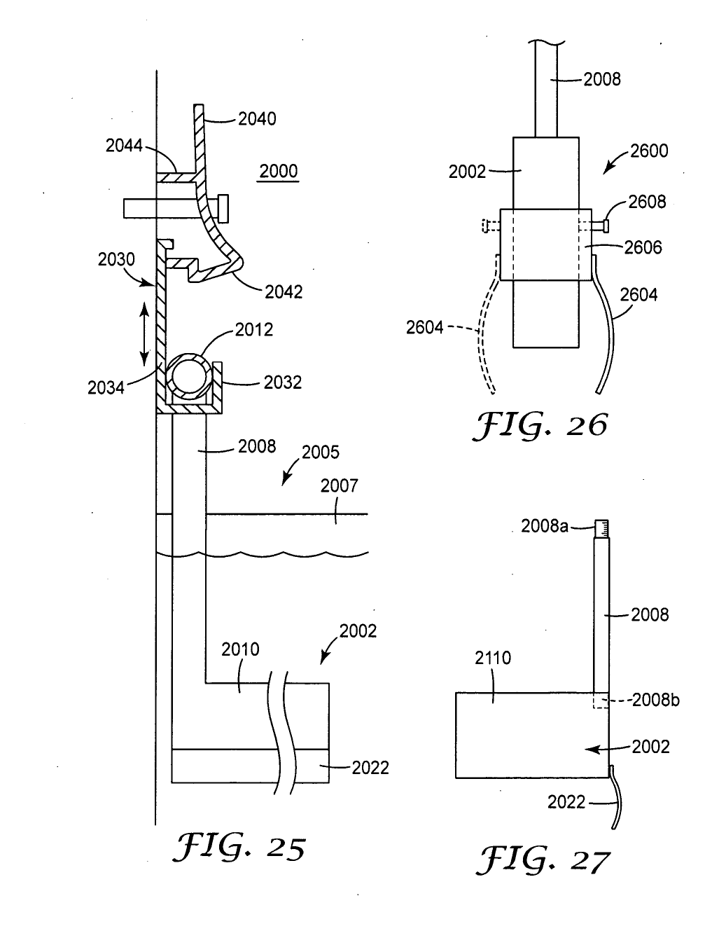

Reference is made to FIG. 25 for illustrating another approach for installing a system to a fryer system. In the system 2000 the central supporting member 2012 is directly connected a fryer system so as to be placed over and across the tops of the frying vats 2005 by one or more brackets 2030 having a construction and relative size as is illustrated. The bracket 2030 includes a channel 2032 into which the central supporting conduit 2012 may be placed for supporting the latter. The bracket 2030 may includes a vertical portion 2034 that is adapted to be held against and slid relative to one of the sidewalls 2007 of a vat. The bracket 2030 may have different configurations and different devices may be used to hold it in place. The bracket 2030 may be individually mounted to a sidewall by a threaded member (not shown) or may be, as is illustrated, held in place and vertically movable with respect to a fryer basket bracket 2040. The fryer basket bracket 2040 includes a horizontal portion 2042 which when assembled engages the bracket 2030 and may include another portion 2044 that is adapted to hold an edge of a fryer basket (not shown). It will be understood that the present description envisions other approaches for securing the conduit 2012 relative to a vat system.

As illustrated in FIG. 26 a probe assembly 2600 includes a probe body 2602 that is provided with a spring-biased electrical conductor element 2604 attached to a slider device 2606. Both the slider device and the conductor element are made of materials for allowing flow of the low voltage generated by the probe to the vat sidewalls. The slider device is also made of a material that is compatible with food. The spring-biased electrical conductor element 2604 may be a spring clip and may have a construction similar to that illustrated or may have other constructions as noted above. The slider device 2606 may be coupled to the slide relative to the probe body 2602 and is secured in a desired location by a set screw 2608 or the like. The slider device 2606 may have a configuration that embraces opposing ends of the probe body 2602. Other spring-biased electrical conductor elements 2604 may be added. The slider device 2606 is versatile since it may be removed and replaced, whereby the conductor element 2604 may be positioned on an opposing sidewall of the probe assembly.

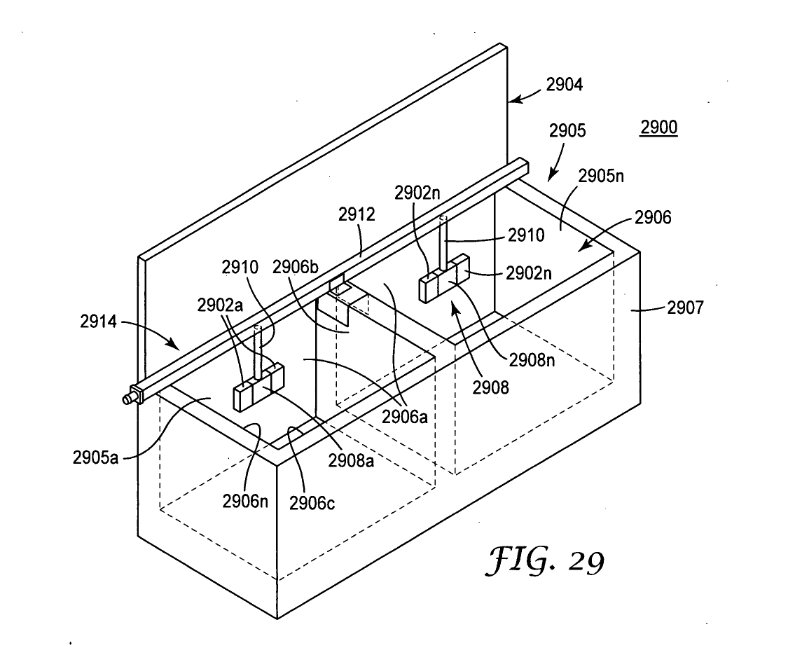

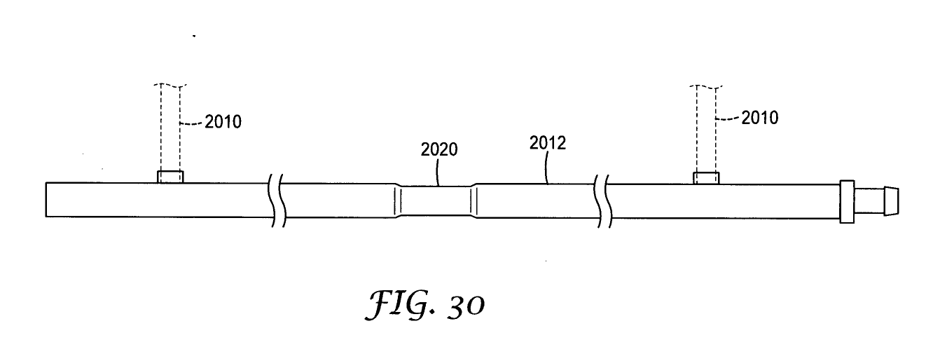

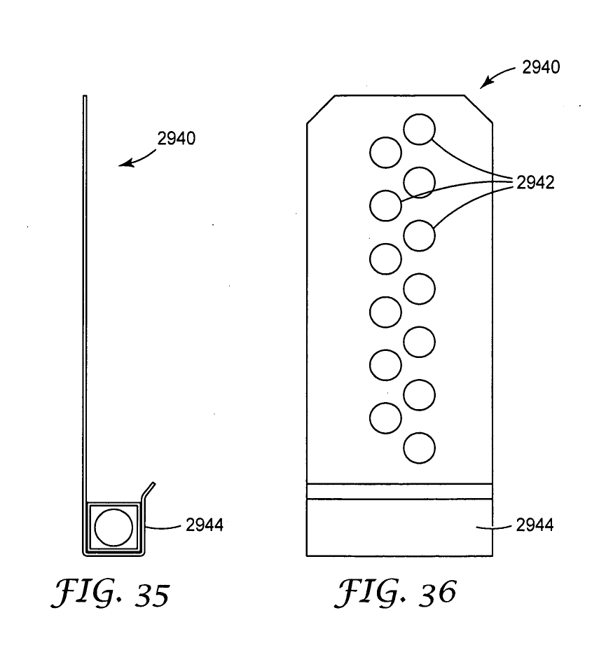

Reference is made to FIGS. 29-36 for illustrating other exemplary embodiments used for installing probe assemblies 2902a-n (collectively, 2902) in a manner that allows for retrofitting in accordance with the present description. As in the previous embodiments, system 2900 facilitates easy retrofitting installation as well as facilitates the cleaning of the vats as well as the probe assemblies themselves.

FIG. 29 illustrates that a system 2900 may include the probe assemblies 2902 used in combination with a food treating system 2904, such as a fryer system 2904 including a plurality of frying vats 2905a-n (collectively, 2905) into which cooking oil media (not shown) may be added to treat, for example, different food items (not shown). Internal walls 2906a-n (collectively, 2906) of each vat are similar those described earlier and generally have an electrically conductive surface over at least a major portion thereof. As noted, the present description is usable in combination with many types of food treating systems, such as cooking/frying systems having different sizes and shapes as well as different operating components (not shown), such as automatic fryer baskets (not shown) that operate therewith.

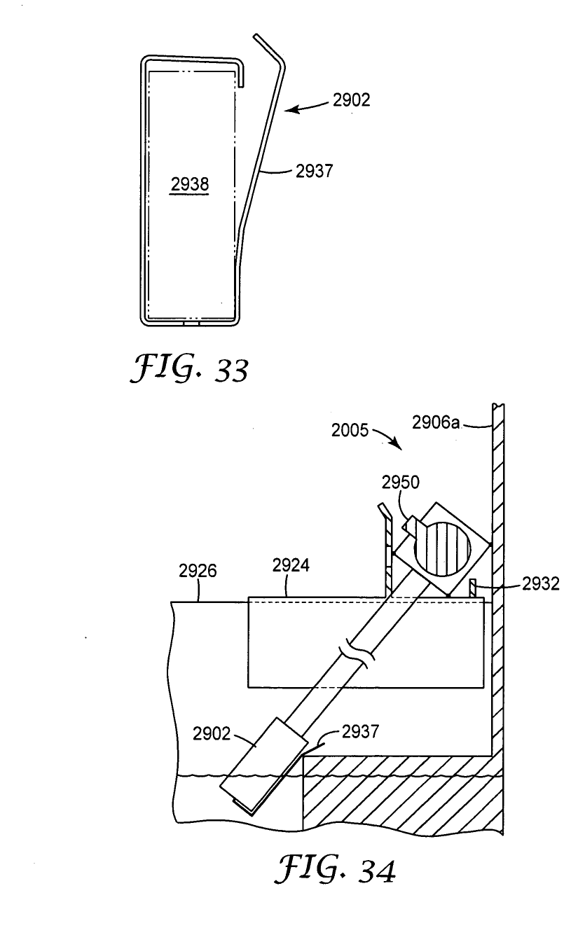

Also, the probe assemblies 2902 may be arranged to be partially or totally submerged (not shown) in cooking oil while being in electrical contact with the internal walls. The probe assemblies 2902 may be similar to those described in earlier embodiments for introducing low voltage to the vat and cooking oil. The probe assemblies 2902, as noted, may have different sizes and configurations, such as, but not limited to, the configurations of the kinds previously described and/or illustrated. The probe assemblies 2902 of the present description are not limited to the sizes and configurations illustrated. As illustrated in FIG. 33, a probe assembly 2902 may be surrounded by one or more spring-biased electrical conductor elements 2937, such as an electrically conductive spring-clip 2937. As in the other exemplary embodiments, the spring-clip 2937 may be releaseably connected to surround a probe body 2938. Accordingly, the spring-biased electrical conductor element 2937 compensate for any misalignments of the probe assemblies not being properly aligned during installation for making contact with the internal wall portions for ensuring transferring the low voltages of the probe assembly thereto for the advantages noted above.

In the present exemplary embodiment, a single probe 2902 may be adapted to cooperate with each one of the frying vats 2005. As illustrated a pair of laterally extending end probe portions 2902a, 2902a; 2902n, 2902n is adapted to cooperate with respective rearward (as viewed in FIG. 29) internal walls 2906a of the vats. While a single probe assembly 2902 is illustrated in each vat, any suitable number of probe assemblies 2902 may be provided. Each probe assembly includes a central holder 2908a-n (collectively, 2908). The central holders 2908 may be a central clamp or clip member made of an electrically conductive material suitable for use with food, such as stainless steel or other similar materials. The central holders 2908 may make contact, along with the probe assemblies 2902 with a rearward internal wall 2906a to distribute electrons consistent with the teachings of the present description. Alternatively, the clips need not be made of an electrically conductive material. The central holders 2908 may be made of hollow ended constructions into which the probe assemblies are fitted. Other constructions, modes of securing, and materials are envisioned for the central holder 2908 to hold or retain the probe assemblies 2902. The central holder 2908 may be threadedly coupled to a coupling tube 2910 that allows the wiring (not shown) from each probe assembly 2902 to pass therethrough into the central supporting member or conduit 2912 and to a controller (not shown) but similar to those described in the above embodiments. The coupling tube 2910 may be threadedly attached at opposing end portions or may be coupled by other suitable approaches including those described above, such as welding and the like.

It will be noted that the probe assemblies 2902 in the illustrated exemplary embodiment are generally centrally disposed in a frying vat 2905. However, the probe assemblies 2902 are adapted to be positioned in other predetermined locations in multiple preexisting frying vats 2905, such that the probe assemblies meet operating conditions that are consistent with the principles of the present description. For example, with the installation system of the present description, the probe assemblies 2902 may be supported in such a manner as to engage outer wall 2907 (FIG. 29) in a similar fashion as probe 950f (described in FIG. 9).

The supporting tubes 2910, which when mounted are generally upstanding, and the supporting member 2912 or conduit member 2912, which when mounted is horizontally disposable serve as part of a supporting assembly 2914 that supports the probe assemblies 2902. The central supporting member 2912 may be constructed with threaded fittings (not shown) at spaced apart linear intervals for threaded cooperation with the supporting tubes 2910 connected to probe assemblies 2902. Such a supporting assembly 2914 includes the supporting tubes 2910 generally extending from the conduit member 2912. In this illustrated exemplary embodiment the supporting member 2912 may be constructed as a polygonal member that, as noted in an earlier embodiment, carries probe wires (not shown) to and from each of the probe assemblies 2902 to the controller (not shown), but similar to those described in the other embodiments. In the illustrated embodiment, the supporting member 2912 may have a generally square shape in cross-section. The supporting member 2912 has at spaced longitudinal intervals centering portions 2920 of reduced cross-section, which cooperate with the supporting member 2912 in a manner to be described for laterally restraining the latter against linear displacement along its longitudinal axis. It will be appreciated that the dimensions and relative sizes of the components is for illustration purposes, for example, the reduced centering portions may be made more shallow than illustrated. While illustrated as a single piece, the supporting member 2912 may be made of joinable components as well.

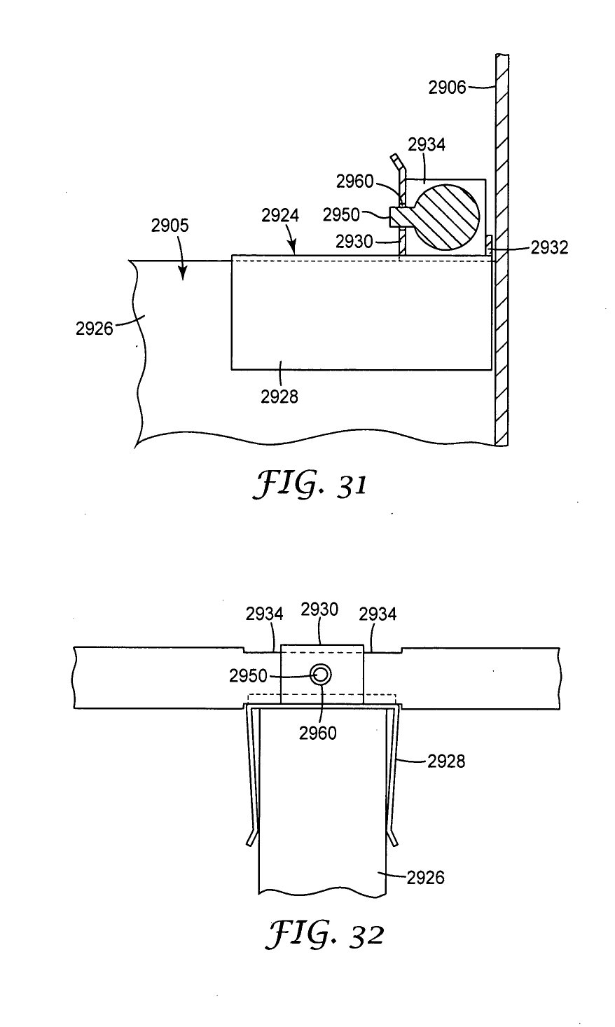

Continued reference is made to FIGS. 29 & 30 as well as FIGS. 31, 32 & 34. The supporting assembly 2914 in one exemplary embodiment includes a mounting bracket member 2924 or the like that is connectable on top of vat partition walls 2926. In one exemplary embodiment, the bracket member 2924 may be of the type that enables the central supporting member 2912 to be rotated about its longitudinal axis as well as centers it against linear displacement. To enable rotation of the supporting member 2912, a user will pick up the latter and rotate it by 90 or 180 degrees in order for the probe assemblies to be correspondingly rotated. As such, rotation enables the probe assemblies 2902 attached thereto to be rotated up and away from the rearward internal wall 2906a. In this fashion, the frying vats 2905 may be more easily cleaned as well as the probe assemblies themselves. A wide variety of brackets and the like may be used for supporting the central supporting member 2912 relative to a vat system. In the present exemplary embodiment the mounting bracket member 2924 may include a generally U-shaped biased clip or clamping portion 2928 as well as a pair of upstanding and spaced part projecting elements 2930, 2932. The clamping portion 2928 is adapted to be mounted on a partition wall 2926 to frictionally engage and retain the probe assemblies 2902 in any position including the illustrated position, whereby the supporting member 2912 is immediately adjacent a rearward internal wall of a frying vat.

As illustrated in FIGS. 1, 29 & 31, the projecting elements 2930, 2932 may be spaced apart by a distance to removably receive the centering portions 2920 of the supporting member 2912. All of a centering portion may be positionable between the projecting elements 2930, 2932. To ensure centering of the horizontally disposable supporting member 2912 its shoulder portions 2934 on opposing sides of the centering portion 2920 (See FIGS. 31, 32) may engage lateral edges of the projecting elements 2930. Also in the illustrated embodiment the projecting element, 2932 may be shorter than the projecting element 2930 to enable rotation of the supporting member or conduit 2912, as illustrated in FIG. 34. In addition, the exemplary embodiment, a welded boss or protrusion 2950 may be attached to the centering portion or even to a non-centering portion for purposes of centering the supporting member or conduit 2912 as the protrusion fits through an opening 2960 in the projecting element 2930. It will be appreciated that one or more of the projecting elements may be relatively flexible to allow insertion, removal, and rotation of the supporting member or conduit 2912 as described in the present embodiments. Further as illustrated in FIG. 34, rotation enables the probe assembly 2902 that may include a spring-clip 2937 to engage internal vat walls of fryers that have offset orientations as indicated. Other locations and numbers of protrusions, similar to protrusion 2950 may be provided. For example, one or more protrusions may be located to engage laterally the projection element 2930. The sizes and the shapes of the protrusions may be altered consistent with teachings of the present description. While lateral centering of the supporting member 2912 is envisioned, the present description envisions that such need not be the case. In that case, a probe assembly may be able to contact another sidewall of the vat. Should two sidewalls be envisioned for being contacted, the probe assemblies described in previous embodiments may be used.

Reference is made to FIGS. 35 & 36 for illustrating another exemplary embodiment of the present description. This embodiment includes a mounting bracket 2940 having a body portion with a plurality of staggered holes or openings 2942. The mounting bracket 2940 is adapted to be held against, for instance, the rearward internal wall by retaining members (not shown). The mounting bracket 2940 has a retaining portion 2944 for removably receiving therein the supporting member 2912. The retaining portion 2944 may also be a spring-clip or clamp. The mounting bracket 2940 may be made of a suitable material for the purposes envisioned. The mounting bracket openings 2942 permits the vertical positioning of the probe assemblies (not shown) relative to the depth of a vat. It will be understood that the mounting bracket 2940 need not be used with the bracket described above.

A method of installing the above system includes measuring a middle of the vat width as viewed in the drawings. The mounting bracket is placed on a vat partition wall and slid against a rearward internal wall. The supporting member 2912 is mounted between the projecting elements 2930, 2932 and slid until the probe assembly 2902 is moved to the center of the vat. As will be apparent, the system 2900 and method of installing the same enable versatile, low cost, and yet easy retrofitting of a fryer system or the like which utilizes multiple probe assemblies 2902.

The above embodiments have been described as being accomplished in a particular sequence, it will be appreciated that such sequences of the operations may change and still remain within the scope of the present description. For example, an illustrated embodiment discusses one set of testing protocols wherein the minimum validation value for the gas monitor must be satisfied before apply testing gas to obtain a first reading. It will be appreciated that such preliminary procedures need not be followed for one to conduct testing of gas sensor assemblies. Also, other procedures may be added.

This present description may take on various modifications and alterations without departing from the spirit and scope. Accordingly, this present description is not limited to the above-described embodiments, but is to be controlled by limitations set forth in the following claims and any equivalents thereof. This present description also may be suitably practiced in the absence of any element not specifically disclosed herein. All patents and publications noted above, including any in the Background section are incorporated by reference into this document in total.

What is claimed is:

1. A method of inhibiting degradation of a food treating medium, the method comprises: providing a container for containing a food treating medium and having at least a conductive portion; and, applying a source of electrons from a low voltage source including a semiconductor material associated with the conductive portion having a food treating medium in contact with the conductive portion.

2. A system of inhibiting degradation of a food treating medium, the system comprises: a container for containing a food treating medium and having at least a conductive portion; and, one or more sources of electrons associated with the conductive portion for applying electrons from a surface of the conductive portion to a food treating medium in contact with the conductive portion.

3. A method of inhibiting degradation of a food treating medium, the method comprises: providing a container having one or more vats for containing a food treating medium and having at least a conductive portion to each of the one or more vats; and, applying electrons by electrically coupling the conductive portion with one or more sources of electrons, independent of the container, for applying electrons from a surface of the conductive portion to a food treating medium in contact with the conductive portion of the one or more vats for inhibiting degradation of a food treating medium.

4. A method of inhibiting degradation of a food treating medium, the method comprises: providing a container for containing a food treating medium and having at least a conductive portion; and, applying electrons by electrically coupling the conductive portion with one or more sources of electrons for applying electrons from a surface of the conductive portion to a food treating medium in contact with the conductive portion, wherein the one or more sources are separate and apart from a food treating medium contained in the container.

5. A food treating method comprising: monitoring at least a quality parameter of a food treating medium to obtain a predetermined value correlated to quality; and applying electrons to the food treating medium in response to the predetermined value so as to inhibit degradation of the food treating medium.

6. A food treating system comprising: a food treating apparatus configured for holding a food treating medium; a monitoring assembly for monitoring at least a quality parameter of a food treating medium to obtain a predetermined value correlated to quality; and a stabilizing assembly operable for applying electrons to a food treating medium in response to the predetermined value so as to inhibit degradation of the food treating medium.

7. A system that comprises: a vessel that includes at least a wall having a portion for defining at least a first chamber and a second chamber divided by a common wall for holding cooking oil; a probe supporting assembly mountable on the common wall for supporting a probe in each of the first and second chambers so that that the probes are within the cooking oil, wherein the probe supporting assembly supports the probes at a position below a container that is insertable into at least of one the first and second chambers.

8. A probe assembly for applying electrons to a cooking oil medium in a vat defined, in part, by a supporting wall including an electrically conductive portion, the probe assembly comprises: a housing assembly; a material within the housing assembly for supplying electrons in response to energization thereof, wherein the material is in electrically conductive relationship with at least a portion of the housing assembly for transferring electrons thereto; and, a coupling assembly on the housing assembly for securing and supporting the housing assembly on a supporting wall, whereby the electron transferring portion is in conductive relationship with the conductive portion of the supporting wall.

9. A system adapted for mounting a plurality of probe assemblies relative to a fryer having a plurality of vats, the system comprises: a plurality of probe assemblies, a supporting assembly for supporting each of the probe assemblies such that one or more the probe assemblies cooperates with at least a respective one of the vats, the supporting assembly is attachable to the fryer; and, the probe assemblies are coupled to the supporting assembly.

10. A method of controlling a plurality of probe assemblies that are adapted to be associated with a fryer system containing a plurality of vats and into which different cooking oil media may be added, the method comprising:
providing a plurality of probe assemblies each of which is independently operated;
supporting each of the probe assemblies on the fryer system so that one or more of the probe assemblies is operatively associated with at least one of the vats; and
controlling operation of the probe assemblies.

11. A method of inhibiting degradation of a food treating medium, the method comprises: providing a container for containing a food treating medium and having at least a conductive portion; and, providing a semiconductor material integrated in the container and associated with the conductive portion and having a food treating medium in contact with the conductive portion.

12. A system of inhibiting degradation of a food treating medium, the system comprises: a container for containing a food treating medium and having at least a conductive portion; and, one or more sources of electrons integrated in the container and associated with the conductive portion for applying electrons from a surface of the conductive portion to a food treating medium in contact with the conductive portion.

13. An installation system for retrofitting a food treating system having one or more vats having sidewalls, the installation system comprising:
a supporting assembly including a generally horizontally disposable supporting member that is adapted to be supported by a vat, and at least one or more generally extending supporting members arranged for depending in a generally upstanding relationship to and along the length of the horizontally disposable supporting member in response to the horizontally disposable supporting member being supported on a vat, wherein each of the one or more extending supporting members is coupled to the horizontally disposable supporting member at one end thereof, and
at least one probe assembly coupled to another end of the one or more extending supporting members and oriented to be engageable with a vat sidewall.
